(19) United States
(12) Patent Application Publication
(10) Pub. No.: US 2026/0063635 A1
(43) Pub. Date: Mar. 5, 2026

(54) TWO-DIMENSIONAL AND NANO-MATERIALS AS MASS TAGS AND CELL LABELING SYSTEMS IN MASS CYTOMETRY AND HIGH-DIMENSIONAL IMAGING

(71) Applicants: DREXEL UNIVERSITY, Philadelphia, PA (US); UNIVERSITÀ DEGLI STUDI DI PADOVA, ITALY, Padua (IT)

(72) Inventors: Yury GOGOTSI, Warminster, PA (US); Lucia Gemma DELOGU, Sassari (IT); Laura FUSCO, Trieste (IT)

(21) Appl. No.: 19/107,117

(22) PCT Filed: Sep. 1, 2023
(86) PCT No.: PCT/US2023/073303

§ 371 Date: Feb. 27, 2025

Related U.S. Application Data
(60) Provisional application No. 63/374,460, filed on Sep. 2, 2022.

(51) Int. Cl. G01N 33/569; G01N 33/68

(52) U.S. Cl. CPC G01N 33/56972; G01N 33/6851; G01N 2458/00

(57) ABSTRACT

Methods that include tagging at least one cell with a MXene, the cell optionally being an immune cell; and detecting at least one component of the MXene using one or more of single-cell mass cytometry by time-of-flight (CyTOF), imaging mass cytometry (IMC), and ion beam imaging (MIBI-TOF). Systems that include a cell tagged with an amount of a MXene; and a detection train configured for at least one of time of flight (CyTOF), imaging mass cytometry (IMC), and ion beam imaging by time-of-flight (MIBI-TOF) that detects the MXene. Methods that include tagging a population of cells with at least one MXene: and processing the population of cells with at least one of time-of-flight (CyTOF), imaging mass cytometry (IMC), and ion beam imaging by time-of-flight (MIBI-TOF) that detects the at least MXene; and relating the detection of the at least one MXene to a characteristic of the population of cells.

### RELATED APPLICATIONS

The present application claims priority to and the benefit of U.S. patent application No. 63/374,460, “Two-Dimensional- And Nano-Materials As Mass Tags And Cell Labeling Systems In Mass Cytometry And High-Dimensional Imaging” (filed Sep. 2, 2022). All foregoing applications are incorporated herein by reference in their entireties for any and all purposes.

### TECHNICAL FIELD

The present disclosure relates to the field of mass cytometry, high-dimensional imaging and to the field of mass tags.

### BACKGROUND

At present, different techniques are used to detect nanomaterials, consisting of labeling methods or, less often, label-free methods. The former requires labels such as radioisotope or fluorescent dyes, which involve time-consuming staining protocols and lead, among several other intrinsic problems, to the risk of tag detachment over time. Instead, the latter approach, based for example on electron microscopy, tomography, and mass spectrometry, allows the detection of nanomaterials by exploiting their intrinsic properties. The foregoing techniques, however, suffer from slow imaging speed, strong background, or weak photoluminescence signals; none of them can achieve nanomaterial detection at the single-cell level resolution simultaneously interrogating dozens of cell types and cell-parameters, ensuring a depth and high dimensionality of the biological data.

As explained below, however, the current state of the art exhibits a number of shortcomings.

### Difficulties in Nanomaterial Detection

The ability to track, detect and quantitatively measure nanomaterials in cells and tissues has driven their increasing exploitation in biomedicine. However, ordinary imaging strategies currently available to detect nanomaterials suffer from several limitations, including slow imaging speed, strong background, or weak signals, and do not allow a label-free trackability at the single-cell level.

There is a need to overcome the limits of current nanomaterial detection techniques, which involve time-consuming functionalization protocols and are not able to simultaneously offer a broad view on cell-specific biological effects. In addition, the material functionalization itself may affect the toxicological profile, cell interaction and uptake capabilities, thus possibly impairing their biomedical effectiveness.

The development of label-free, high-resolution and high-dimensional approaches that simultaneously visualize nanomaterials in multiple cell types, thus enabling insight into cell functions and interactions, together with their spatial localization in tissues, is crucial for translating nanomaterials to clinical applications.

### Difficulties in Multiple Live Cell Labeling Strategies Compatible with CyTOF, IMC, MIBI-TOF for In Vitro and In Vivo Experiments

Tracking and labeling of cells and biological moieties using biocompatible chemistries over a certain period of time is vital in biology and medicine, e.g. to understand cell interactions, for drug screening, precise diagnosis and treatment, such as monitoring cell survival and tissue regeneration after stem cell transplantation; understanding the immune responses; invasion and metastasis of cancer, etc. Over the past two decades, cell labeling strategies have remained the same, with the same technological limitation: a lack of chemical versatility. To date, novel methods are tremendously needed in basic research as well as translational medicine to enable cell tracking in single-cell mass cytometry.

### Need to Expand and Fully Exploiting the Available Mass Cytometry and High-Dimensional Imaging Detection Channels

Single-cell multiplexed analysis is crucial to effectively explore the heterogeneous cell populations in biological samples. Single-cell mass cytometry by time-of-flight (CyTOF) is already replacing flow cytometry worldwide. This well-established technique is based on mass spectrometry to detect metal element-tagged probes, thus allowing for parameter discrimination according to their mass/charge ratio (m/z), with minimal overlap or signal background. Compared with fluorescence flow cytometry, CyTOF offers increased resolution and parameterization of simultaneous dimensions. Whereas CyTOF is limited to cells in suspension, such as circulating blood cells or cells dissociated from tissues, imaging mass cytometry (IMC) and multiplexed imaging by time-of-flight (MIBI-TOF) similarly use elementally labeled probes to measure dozens of molecular parameters at single-cell resolution in situ. These techniques are becoming increasingly prevalent and have greatly advanced diverse fields including immunology, oncology, neuroscience, and infectious diseases.

Although the newest time-of-flight mass spectrometers can simultaneously discriminate 135 channels in the atomic mass range of 75 to 209 Da, over 60% of the isotope channels have not been implemented due to the lack of available chemistries containing different metals or heavy metal isotopes. Therefore, developing new chemistries for time-of-flight mass spectrometers is key to expanding and fully exploiting the available detection channels to further improve the parameterization for single-cell multiplexing and spatial imaging of tissues.

### Barcoding Systems

CyTOF enables the multidimensional study of complex samples never achieved before. However, the full potential of this technology is challenged by the cost and time needed to analyze a high number of samples. To overcome this issue, barcoding strategies based on direct labeling of cells have been developed enabling staining and analysis of a single, combined sample. Sample multiplexing also minimizes batch variation, enables scaled-up experiments, and improves data consistency and workflow efficiencies. However, current barcoding shows some inherent problems, mainly associated with signal intensity and limited number of barcodes to use at a time, live cell barcoding only for immune cells, and the need of protocols based on fixation and permeabilization. Accordingly, there is a long-felt need for a platform technology that can address the various shortcomings identified above.

### SUMMARY

The present disclosure relates to, inter alia, the detection of 2D materials such as MXenes by single-cell mass cytometry by time-of-flight (CyTOF), imaging mass cytometry (IMC) and ion beam imaging by time-of-flight (MIBI-TOF), their chemical characterization, quantification, and their use as mass tags.

MXenes are a family of two-dimensional metal carbides and nitrides such as Ti₂C, Mo₂C, Ti₃C₂, and the like. As used herein, the term “mass tags” here refers to tools useful in bioimaging, e.g., for labeling of cells, tissues, subcellular organelles, or biomolecular targets of interest with MXenes or other nanomaterials (with a chemical composition in atomic mass that fits the detection range of CyTOF, CyTOF-XT, IMC and MIBI-TOF). The MXenes and other nanomaterials can be detected and quantified at the single-cell and tissue level for a wide variety of biomedical applications, including those that require information about their biodistribution, fate and localization. These mass tags can be used in a range of applications, including labeling, detection, identification, and tracking of cells, in addition to other applications such as cell barcoding systems, spatially resolved biological targets in tissues, and detection of biological markers with single-cell resolution. In particular, the disclosed technology can be used in, e.g., immunology and regenerative medicine for (i) multi-imaging agents for cell-tracking and (ii) detection of weakly expressed antigens or rare cell populations. The disclosed technology can be used to determine whether a certain cell process is occurring (or not occurring), the distribution of cells in a tissue, to determine uptake of the MXene tags, and the like. One can evaluate docking of MXene tags experimentally and/or via simulation.

The disclosed technology has a number of applications, including (1) the detection of MXenes and other materials with similar chemical characteristics in cells and tissues while simultaneously surveying a high degree of biological information from single samples; and (2) using the detection of the above-mentioned materials to, e.g., label, identify, track, and barcode cells by CyTOF, IMC and MIBI-TOF and other mass spectrometry-based tools. Among other advantages, the disclosed technology is compatible with all the commercial panels of metal-tagged antibodies for CyTOF, which provides an efficient way of tracking the materials together with several biological information; the method can also be used to conjugate drugs. The disclosed technology also allows material tracking on a high number of cell types at the same time, within tissues and at single-cell level. The disclosed technology can also be adapted for other 2D materials useful for biomedical applications based on their masses.

Detecting 2D materials along with an efficient uptake of the material and biocompatibility in cells and in vivo has many advantages for their applications as mass tags for, e.g., (i) cell labeling and tracking tools, (ii) cell barcoding system, and (iii) the detection if conjugated with specific antibodies of low expression markers.

Another strategy for multiplexed labeling is based on green fluorescent protein and as such require genetic manipulation of the cells, which is prohibitive for many types of applications. For non-genetic labeling, carboxyfluorescein succinimidyl ester (CFSE) is the most commonly used agent, and it does not allow for versatile applications in CyTOF, IMC, MIBI-TOF or for many cell types as the disclosed technology can do.

Further, none of the present commercially available tools are able to barcode live cells, while being compatible with FIX and PERM cell protocol, the disclosed technology can overcome current limitation, expanding the CyTOF detection range, being universally available for any cell type, and reducing the cost of limitation of antibody conjugation (e.g. CD45 for current commercially available kit). Efficient barcoding can speed sample analysis and reduce costs of the sample run. Additionally, the number of detectable atoms in a MXene can be from 300 to 1000 higher than in current mass tags, allowing the detection of very low expressed markers when conjugated with specific antibodies. Clinically relevant markers with prognostic value are an advantage of the disclosed technology.

The disclosed technology can be applied in a number of applications. As a first example, the technology can be applied to single-cell labeling, detection, and tracking for multiple populations which can be the first of its kind in the market compatible with single-cell mass cytometry, imaging mass cytometry, and ion beam imaging. These kits will overcome the current limitation of green fluorescent protein and CFSE cell labeling and cell proliferation tracking.

As a second example, the disclosed technology can be used in methods for mass cytometry cell barcoding MXene-based kits, together with software for cell de-barcoding. These kits overcome the limitation of current commercial kits: they are compatible with fixation and permeabilization protocols, they are compatible with live cells, and they can also be an antibody-free system.

As a third example, the disclosed technology can be applied to MXene-based kits. Such kits can be used for the detection of low expressed clinically relevant markers, thanks to conjugation with specific antibodies.

The present disclosure provides, for example, a method, comprising: tagging at least one cell with a MXene, the cell optionally being an immune cell; and detecting at least one component of the MXene using one or more of single-cell mass cytometry by time-of-flight (CyTOF), imaging mass cytometry (IMC), and ion beam imaging (MIBI-TOF).

Also provided is a system, the system configured to perform a method of the present disclosure, for example, a method according to any one of Aspects 1 to 13.

Further disclosed is a system, the system comprising: a cell tagged with an amount of a MXene; and a detection train configured to perform on the cell at least one of time of flight (CyTOF), imaging mass cytometry (IMC), and ion beam imaging by time-of-flight (MIBI-TOF) that detects the MXene.

Additionally provided is a method, the method comprising: tagging a population of cells with at least one MXene; and processing the population of cells with at least one of time-of-flight (CyTOF), imaging mass cytometry (IMC), and ion beam imaging by time-of-flight (MIBI-TOF) that detects the at least MXene; and relating the detection of the at least one MXene to a characteristic of the population of cells.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various aspects discussed in the present document. In the drawings:

FIG. 1. “Label-free tracking of 2D matErials by mass cytometry and MIBI-TOF design” (LINKED) approach. a, Selection of CyTOF-visible elements, shown in grey in the periodic table, to synthetize biocompatible MXenes: Nb₄C₃, Mo₂Ti₂C₃, and Ta₄C₃; a schematic representation for their detection on cell and tissues using three mass cytometry-based methods (single-cell mass cytometry, imaging mass cytometry, and ion beam imaging) is also shown. b, Mass cytometry permits the detection of stable isotope masses from 75 Da to 209 Da. The colored pics represents all current metal tags used on CyTOF. MXenes here designed by LINKED were inserted in unexplored channels. Nb₄C₃, Mo₂Ti₂C₃, and Ta₄C₃ detection is permitted in the ⁹³Nb, ⁹⁵Mo and ¹⁸⁰Ta channel, respectively. c, MXene compatibility with available commercial tags used in the present study: a total of 48 isotopes have been used for human ex vivo and ex vivo and in vivo mouse experiments. A total of 76 mass cytometry probes have been used (56 for human and 21 for mice) for the assessment of viability, cytokine detection, markers for cluster of differentiation (CD), palladium and cadmium-based barcoding, DNA staining, and biotin detection.

FIG. 2. MXenes tracking in human PBMCs at the single-cell level. a-f, PBMCs were incubated with 50 μg/mL of Ti₃C₂, Nb₄C₃, Mo₂Ti₂C₃ or Ta₄C₃ for 24 h and stained for mass cytometry analysis. Representative tSNE for each MXene showing the single-cell signal intensity compared to the control (Ctrl); intensity is represented from blue (low interaction) to red (high interaction) (a). Heat maps reporting the MXenes median intensity for each specific channel in all the major immune cell subpopulations identified (b-d). Grouped bar plots showing Nb₄C₃, Mo₂Ti₂C₃ and Ta₄C₃ median intensity (e) and % of positive cells for all the replicates in all PBMC subpopulations identified (f). g, MXenes detection in human PBMCs by imaging mass cytometry (IMC) Panels I represent IMC images reporting in green the single-cell tracking of Nb₄C₃, Mo₂Ti₂C₃ or Ta₄C₃. Panels II represent IMC images reporting in blue and light blue the DNA signal. Panels III represent IMC images reporting a composite image of DNA (blue) and Nb₄C₃, Mo₂Ti₂C₃ or Ta₄C₃ (green).

FIG. 3. Analysis of cell viability versus MXene single-cell binding, single-cell cytokine analysis and RNA sequencing on human PBMC subpopulations. a-c, PBMCs were treated with 50 μg/mL of Ti₃C₂, Nb₄C₃, Mo₂Ti₂C₃ or Ta₄C₃ for 24 h. Spider charts represent the impact on viability (blue), expressed as Cisplatin (Cis) median intensity, vs the MXene tracking (orange) expressed for fifteen immune cell types after exposure of PBMCs to 50 μg/mL of Nb₄C₃ (a), Mo₂Ti₂C₃ (b) or Ta₄C₃ (c) for 24 h. d, Cell functionality analysis of PBMCs by Luminex technology. Heat map shows the hierarchical cluster analysis of inflammatory mediators released by PBMCs treated with 50 μg/mL of Ta₄C₃, Mo₂Ti₂C₃ or Nb₄C₃ for 24 h. As positive control, cells were exposed to LPS (2 μg/mL). Association clusters for samples are represented by dendrograms at the top of the heat map. Individual cytokines were colored according to z-scored normalized value (blue=down-regulated, yellow square=unmodulated cytokines, red=up-regulated cytokines). Analytes represented are the ones passing the FDR p value of 0.05 by fitting a general ANOVA model between control and all the experimental conditions. e,f, Gene expression analysis by mRNA-Seq. Heatmap of all differentially expressed genes between ConA, LPS, Ta₄C₃, Mo₂Ti₂C₃ and Nb₄C₃ vs. control using FDR<0.01; classic LPS-induced transcripts (IL1AB, IL10, and IL6) associated with monocyte activations are represented (e). Numbers of differentially expressed genes based on different cutoff of p-adjusted FDR value between experimental conditions and control (f).

FIG. 4. MXene cocktail on human PBMCs: viability and detection. a-g, PBMCs were incubated with a cocktail of N₄C₃—260 nm, Mo₂Ti₂C₃—240 nm and Ta₄C₃—160nm (50 μg/mL each) for 24 h and stained for mass cytometry analysis. Cell viability was analyzed using Cisplatin (Cis) reagent by CyTOF (a). Heat maps reporting the MXene median intensity for each specific channel in all the major immune subpopulations identified (b). Bar plots representing the percentage of positive cells to MXenes in total PBMCs after treatment for 24 h with the cocktail of MXenes with different size (Nb₄C₃—150 nm, Mo₂Ti₂C₃—370 nm and Ta₄C₃—810 nm, 50 μg/mL each) or similar size (Nb₄C₃—260 nm, Mo₂Ti₂C₃—240 nm and Ta₄C₃—160 nm, 50 μg/mL each) (c). d-g, PBMCs were incubated with a cocktail of Nb₄C₃—260 nm, Mo₂Ti₂C₃—240 nm and Ta₄C₃—160 nm (50 μg/mL each) for 24 h. Detection in 16 immune cell types was analyzed by CyTOF. Spider chart represents the MXene detection in fifteen immune cell types expressed as percentage of positive cell for each immune cell subpopulation (d). tSNE analysis reporting all the major immune subpopulations identified (e). MXene mean signal intensity (MI) detected per single cell (f). tSNE overlay representation of MXenes MI in immune cell subpopulations (g).

FIG. 5. Assessment of MXene cocktail impact on T cells-dendritic cells by a CyTOF based enzymatic labelling LIPSTIC. a, Schematic representation of DC treatment and analysis (upper panel). DCs were treated with a cocktail containing 50 μg/mL of each MXene and cell uptake of each material was monitored by CyTOF analysis, thanks to their detectable masses (⁹³Nb, ⁹⁵Mo, and ¹⁸⁰⁻¹⁸¹Ta). Schematic representation of Labelling Immune Partnerships by SorTagging Intercellular Contacts (LIPSTIC) approach (lower panel). The transpeptidase Sortase A (SrtA) and its target motif (G5) consisting in five N-terminal glycine residues are genetically fused to a receptor and a ligand of interest (in this case, CD40L and CD40). When a biotinylated substrate (i.e., a peptide containing LPETG sequence) is provided, it transiently binds the SrtA via formation of an acyl intermediate. Upon interaction between the cells expressing the receptor and ligand, the SrtA catalyzes the transfer of the substrate onto the G5 motif. As the acceptor cell retains the label even after cells separate, the history of interaction is revealed by the presence of the biotinylated label. Experimental set-up: DCs and CD4⁺ T cells were respectively isolated from Cd40^{G5/G5} and Cd40lg^{SrtA/Y} mouse spleens. DCs were treated with Mxenes for 24 h, then further incubated with freshly isolated T cells for 24 h. b, Representative dot plots showing the uptake of Mxenes by DCs following exposure to the MXene cocktail. The control was left in complete medium. c, Dot plots representing LIPSTIC labelling in CD4⁺ T cells (left) and DCs (right). The negative control (UNT) was not provided with the antigen nor treated with the nanomaterials; the positive control (CTRL) was provided with the antigen, but not the nanomaterials; the sample (MXene cocktail) was treated with Mxenes and supplemented with the antigen. d, Grouped bar plots reporting the percentage of biotin⁺ DCs and CD4⁺ T cells. All the experiments were performed in duplicate and shown as mean±SD (One-way ANOVA).

FIG. 6. In vivo biodistribution of MXene cocktail at the tissue and single-cell level. C57B/6j male mice (n=3) were injected I. V. with 20 mg/kg of a cocktail of MXenes (Nb₄C₃, Mo₂Ti₂C₃ and Ta₄C₃) for 24 h. Subsequently, mice were scarified and blood, liver, lungs, and spleen where harvested and stained for mass cytometry analysis. a, Representative circle chart reporting the total MXenes cocktail (Nb₄C₃, Mo₂Ti₂C₃ and Ta₄C₃) signal intensity detected per organ. b, Signal intensity deconvolution for each MXene in all the organs analyzed. c MXenes MFI signal intensity detected in each immune cell subpopulation analyzed per organ. d, MIBI-TOF imaging of tissues from control and MXene-injected mice stained with the indicated metal-labeled antibodies. MXene signal (purple) is detectable across all tissues in the Niobium (⁹³Nb) and molybdenum (⁹⁵Mo) channels. Scale bar—50 μm.

FIG. 7. Material chemical characterization. a-d, Dynamic light scattering (DLS) of Ti₃C₂Tₓ (a), Nb₄C₃Tₓ (b), Mo₂Ti₂C₃Tₓ (c) and Ta₄C₃Tₓ (d) indicating their hydrodynamic radius. e-h, Scanning electron microscopy (SEM) images of Ti₃C₂Tₓ (e), Nb₄C₃Tₓ (f), Mo₂Ti₂C₃Tₓ (g) and Ta₄C₃Tₓ (h) showing the flake size and morphology after delamination.

FIG. 8. Example, non-limiting study workflow. Study workflow showing MXene synthesis, physical-chemical and basic biology characterization (i); ex vivo assessment of MXene detection on 15 human immune cell types by single-cell mass cytometry and imaging mass cytometry and impact of lateral size and as a cocktail of MXenes (ii); MXene evaluation for cell viability, cell functionality and dynamics of cell-cell interactions by means of single-cell mass spectrometry and LIPSTIC approach¹ (iii); and in vivo MXene testing from biodistribution to in vivo cell tracking by single-cell mass cytometry and multiplexed ion beam imaging (iv).

FIG. 9. Impact of Ti₃C₂, Nb₄C₃, Mo₂Ti₂C₃ and Ta₄C₃ on human PBMC viability. a, PBMCs were treated with different concentrations (12.5, 25, 50 and 100 μg/mL) of Ti₃C₂, Nb₄C₃, Mo₂Ti₂C₃ or Ta₄C₃ for 24 h and cell viability was analysed using the LIVE/DEAD® Viability/Cytotoxicity Kit discriminating live from dead cells by simultaneously staining with green-fluorescent calcein-AM to indicate intracellular esterase activity and red-fluorescent ethidium homodimer-1 to indicate loss of plasma membrane integrity. Plasma membrane integrity and esterase activity were measured by using TECAN fluorescence microplate reader. b, PBMCs were treated with different concentrations (25, 50 and 100 μg/mL) of Ti₃C₂, Nb₄C₃, Mo₂Ti₂C₃ or Ta₄C₃ for 24 h and cell viability was analysed by flow cytometry using Fixable Viability Dye 780 staining.

FIG. 10. Immune cell subpopulations gating strategy. Dot plots showing the gating strategy used for the identification of the different immune cell subpopulations by CyTOF.

FIG. 11. Single-cell tracking analysis of MXenes on human PBMC subpopulations. a-l, viSNE distribution of CD4+ Th. cells, CD8+ CT. cells (a-d), B cells (e-h) and CD3− CD19− cells (i-l). Plots representing the use of viSNE to obtain a comprehensive single-cell view of the PBMC subpopulations treated with Nb₄C₃, Mo₂Ti₂C₃ or Ta₄C₃. showing the median intensity of Niobium (⁹³Nb), Molybdenum (⁹⁵Mo) and Tantalum (¹⁸¹Ta) signal in CD4+ Th. cells, CD8+ CT. cells, B cells, monocytes, mDCs and NKs. A representative viSNE graph is shown out of three biological replicates.

FIG. 12. TEM Imaging. Representative TEM images of Nb₄C₃, Mo₂Ti₂C₃ and Ta₄C₃ interactions with PBMCs. Cells were incubated with Nb₄C₃, Mo₂Ti₂C₃ or Ta₄C₃ (50 μg/mL) for 24 h. Arrows in higher magnification micrographs indicate internalized Nb₄C₃, Mo₂Ti₂C₃ and Ta₄C₃. As shown in panel the representative images depict large aggregation and giant vacuoles inside the cells. Scale bars: 0.5, 1 and 2 μm.

FIG. 13. Single-cell impact of Ti₃C₂ and Nb₄C₃, Mo₂Ti₂C₃, and Ta₄C₃ on PBMC viability. Histograms showing Cis median intensity in the different immune cell subpopulations after treatment with Ti₃C₂, Nb₄C₃, Mo₂Ti₂C₃ or Ta₄C₃ and analysed by CyTOF. All the experiments were performed in triplicate and shown as means±SD (Two-Way ANOVA and student T Test).

FIG. 14. Analysis by t-SNE of cell viability upon MXene treatment on human PBMC subpopulations. PBMCs were treated with 50 μg/mL of Ti₃C₂, Nb₄C₃, Mo₂Ti₂C₃ or Ta₄C₃ for 24 h.

FIG. 15. Single-cell cytokine analysis on human PBMC subpopulations. Heat maps showing median marker intensity of INFy, IL2, IL4, IL5, IL17a, IL17f, IL6, MP1β, TNFα, Perforin and Granzyme B (GrB) for gated immune cell subpopulations after treatment of PBMCs with 50 μg/mL of Ti₃C₂, Nb₄C₃, Mo₂Ti₂C₃ or Ta₄C₃ for 24 h. Upregulated (red squares) or downregulated (blue squares) proteins are indicated. Significantly modulated values are marked as bold squares. All the experiments were performed in triplicate and shown as Log₂ Fold Change as compared to the negative control (Two-way ANOVA and Bonferroni post-test).

FIG. 16. Gene expression analysis (RNA-seq on PBMCs). a, Principal component analysis based on the PBMC full normalized RNA-seq gene expression matrix. b, VennDiagram of differentially expressed genes using FDR<0.01. c, Heatmap of differentially expressed genes (n=21) common to Ta₄C₃, Mo₂Ti₂C₃ and Nb₄C₃ using FDR<0.01, as represented in panel b. c,d, Enriched pathways associated with DEGs common to Ta₄C₃, Mo₂Ti₂C₃ and Nb₄C₃ using FDR<0.05 (d) and FDR<0.01 (e). Experiments are performed in triplicates. Genes raw count data was normalized with EDAseq followed by quantile normalization and log2 transformation. Differentially expression analysis was performed using LIMMA. Enriched IPA canonical pathways using deferentially expressed genes coherently modulated by Ta₄C₃, Mo₂Ti₂C₃ and Nb₄C₃ vs controls with FDR<0.05 (d) and FDR<0.01 (e); enrichment p value is represented by the orange line, dotted lines represent two p values cut-offs (p=0.05=−Log₁₀(p)=1.3, and 0.005, −Log₁₀(p)=2.3). Histograms represent the proportion (%) of DEG upregulated (red) or downregulated (green) between MXenes and controls. The circles represent the inferred activation state. The bases of this inferred activation state are literature-derived relationships between genes and the corresponding biological function. Pathways that are activated in MXenes vs controls are marked with a red circle, indicating a positive activation score, whereas pathways inhibited in MXenes vs controls are marked with a blue circle, indicating a negative activation score. For canonical pathways with a gray circle, no sufficient literature-derived information exists to estimate the activation state.

FIG. 17. Analysis of PBMC activation. a, Histogram plots showing the percentage of positive cells for CD25-PE and CD69-FITC staining evaluated by flow cytometry in PBMCs treated with 50 μg/mL of Ti₃C₂, Nb₄C₃, Mo₂Ti₂C₃ or Ta₄C₃ for 24 h. LPS (2 μg/mL) was used as positive control. b, Representative flow cytometry analysis for CD25-PE and CD69-FITC staining of PBMCs and monocytes treated with 50 μg/mL of Ti₃C₂, Nb₄C₃, Mo₂Ti₂C₃ or Ta₄C₃ Data are presented as mean±SD of three independent samples. Statistical differences: *p<0.05; **p<0.01; ***p<0.001 (One-way ANOVA, followed by Tukey's post hoc multiple comparison).

FIG. 18. Characterization of MXenes with similar lateral size. a,b, X-ray diffraction (XRD) of precursor MAX phases (a) and MXenes (b) after topochemical synthesis. The (002) peak shifts to the left, and only (001) peaks remain, indicating successful synthesis and delamination. c-f, Dynamic light scattering (DLS) of Ti₃C₂Tₓ (c), Nb₄C₃Tₓ (d), Mo₂Ti₂C₃Tₓ (e) and Ta₄C₃Tₓ (f) indicating their hydrodynamic radius. g-j, Scanning electron microscopy (SEM) images of Ta4C3Tx (g), Mo₂Ti₂C₃Tₓ (h), Nb₄C₃Tₓ (i) and Ti₃C₂Tₓ (i) showing the flake size and morphology after delamination.

FIG. 19. CyTOF detection of MXenes with similar lateral size in human immune cells. a, Table showing the main physicochemical characteristics of MXenes with similar lateral size (Nb₄C₃—260 nm, Mo₂Ti₂C₃—240 nm and Ta₄C₃—160 nm). b-g, PBMCs were incubated with 50 μg/mL of Nb₄C₃—260 nm, Mo₂Ti₂C₃—240 nm or Ta₄C₃—160 nm and stained for mass cytometry analysis. Cell viability after 24 h was analysed using Cisplatin (Cis) reagent by CyTOF (b). Percentage of cells positive to Nb₄C₃, Mo₂Ti₂C₃, or Ta₄C₃ is reported as bar graphs for total PBMCs (c) and all PBMC subpopulations identified (d). Time course of MXenes uptake in PBMCs treated for 1, 6 and 24 h (e). f,g, Median intensity of Nb₄C₃—260 nm, Mo₂Ti₂C₃—240 nm and Ta₄C₃—160 nm signals after 24 h is also reported as histogram for total PBMCs (f) and all populations (g).

FIG. 20. MXene cocktail on human PBMCs: viability and detection. a,b, PBMCs were incubated with a cocktail of Nb₄C₃—260 nm, Mo₂Ti₂C₃—240 nm and Ta₄C₃—160 nm (50 μg/mL each) for 24 h and stained for mass cytometry analysis. Histograms representing the mean intensity of MXenes in total PBMCs after treatment for 24 h with the cocktail of MXenes with different size (Nb₄C₃—150 nm, Mo₂Ti₂C₃—370 nm and Ta₄C₃—810 nm, 50 μg/mL each) or similar size (Nb₄C₃—260 nm, Mo₂Ti₂C₃—240 nm and Ta₄C₃—160 nm, 50 μg/mL each) (a). Detection on immune cell types was analysed by CyTOF. Spider chart represents the impact on viability (orange), expressed as LD median intensity for dead cells vs the MXene detection in fifteen immune cell types expressed as log₂ fold change of mean intensity (b).

FIG. 21. MXene quantification determined by CyTOF. a, Number of ⁹³Nb, ⁹⁵Mo and ¹⁸¹Ta atoms per cell determined from direct atom analysis in all PBMC subpopulations after the 24 h treatment of PBMCs with a cocktail of Nb₄C₃—260 nm, Mo₂Ti₂C₃—240 nm and Ta₄C₃—160 nm (50 μg/mL each). b, Dynamic range of ⁹³Nb, ⁹⁵Mo and ¹⁸¹Ta determined by CyTOF solution mode. c, mass spectrum of Nb₄C₃, Mo₂Ti₂C₃ or Ta₄C₃ determined by CyTOF solution mode, lanthanide element calibration beads were applied as controls.

FIG. 22. After singlets' and live/dead discrimination, T cells were identified as MHCII⁻, CD4⁺ cells, while DCs as MHCII⁺, CD4⁻, CD11c⁺, B220⁻. CD69 was implemented as a marker of T cell activation. LIPSTIC labelling was then assessed in both cell populations.

FIG. 23. In vivo biodistribution of MXene cocktail at the tissue and single-cell level. a, Percentage of positive cells subtracted to control for all immune cell subpopulations identified per organ reported as bar graphs. b, MXene mean intensity for all immune cell subpopulations identified per organ reported as histograms. Data are presented as mean±SEM among the replicates.

FIG. 24. In vivo biodistribution of MXene cocktail. Representative contour plot of gated CD45+ cells, isolated from liver of MXene-treated mice.

FIGS. 25 and 26 provide example illustrations of the disclosed technology. FIG. 25 provides a Gating strategy for the identification of CD4⁺ CD45.1⁺ T cells and CD4⁺ CD45.2⁺ T cells. Lymphocytes were identified based on Ir191-DNA1 and Ir193-DNA2 signals. Singlets were identified based on event length, while live cells were identified as Pt194-Cisplatin negative events. In live cell population, CD4⁺ T cells were defined as Yb172-CD4 positive and Bi209-MHC-II negative events. CD45.1⁺ and CD45.2⁺ T cell population were identified based on Sm147-CD45.2 and Eu153-CD45.1 detection. FIG. 26 provides example MXene T cell labeling in vivo. a, Schematic representation of the experimental set-up. CD4⁺ T cells were isolated from CD45.1/1 OT-II transgenic mice, treated ex vivo for 24 h with a combination of MXenes, labeled with CFSE, and transferred into C57BL/6J recipient mice. Six hours post T cell transfer, mice were immunized in the footpad with Ovalbumin precipitated in alum. Popliteal lymph nodes were harvested and analyzed by CyTOF 24 and 60 h post immunization. b, MXenes retainment by in vivo proliferating T cells. Contour plots showing CyTOF detection of Nb93, Mo95 and Ta181 versus CFSE-Gd160 in CD4⁺ CD45.1⁺ T cells untreated (t=24 and 60 h after T cell transfer) and MXene treated (t=60 h after T cell transfer). Quadrant gates identify divided cells MXene positive (upper left); undivided cells MXene positive (upper right); undivided cells MXene negative (lower right); divided cells MXene negative (lower left). c, MXene shedding in vivo. Contour plots showing CyTOF detection of Nb93, Mo95, and Ta181 versus CFSE-Gd160 in CD4⁺ CD45.2⁺ T cells from mice receiving MXenes treated CD4⁺ CD4⁺ CD45.2⁺.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

The present disclosure relates to the detection of 2D materials in mass cytometry, imaging mass cytometry and ion beam imaging. As a first demonstration we used transition metal carbides, nitrides and carbonitrides (MXenes) by single-cell mass cytometry by time-of-flight (CyTOF), imaging mass cytometry (IMC) and ion beam imaging by time-of-flight (MIBI-TOF), their atomical chemical characterization, quantification, and their use as mass tags. MXenes are emergent hydrophilic 2D materials with more than 30 stoichiometric compositions and at least 20 solid solutions have been reported. The term “mass tags” here refers to tools for bioimaging such as the labeling of cell, tissues, subcellular organelles, or biomolecular targets of interest with specific MXenes or other nanomaterials with a chemical composition in atomic mass that fits the detection range of CyTOF, CyTOF-XT, IMC and MIBI-TOF.

Some features were considered when designing some these mass tags. First, we wanted the materials to fit the detection range of CyTOF and MIBI-TOF. Next, we aimed to avoid any overlap with channels commonly used for metal conjugated antibodies.

The specific atomic masses of the designed MXenes containing specific transition metals enable their detection by using three different mass cytometry techniques also in combination with commercial mass cytometry probes (antibodies). MXenes are biocompatible and can be detected without chemical functionalization, also in conjunction with conventionally used metal-labeled antibodies and/or other agents.

As a first demonstration, we produced Nb₄C₃, Mo₂Ti₂C₃ and Ta₄C₃ MXenes as eligible candidates for MXene mass tags, which could be naturally identified in the niobium (⁹³Nb), molybdenum (^{92, 94, 95, 96 97, 98, 100}Mo) and tantalum (¹⁸⁰⁻¹⁸¹Ta) channels, respectively, without the need of functionalization with antibodies. The materials had an average lateral size range between 160 and 260 nm and a number of detectable metal atoms between 1.1 and 3×10⁶.

The mass spectra of Nb₄C₃ and Ta₄C₃ determined with CyTOF solution mode had a CyTOF intensity of 2×10⁵ and more than 5×10⁵, while the commercial lanthanide element calibration beads applied as controls have a value lower than 0.5×10⁵.

For Mo, we could identify the 7 different isotopes used as precursors of MXenes in MAX phase. This method also allows the insertion of novel channels such as ⁹³Nb and ⁹⁵Mo channels not previously explored for commercial mass cytometry tags.

The present disclosure also allows live-cell labeling. As a first demonstration, we have detected Nb₄C₃, Mo₂Ti₂C₃, and Ta₄C₃ in human peripheral blood mononuclear cells (PBMCs) from healthy human donors. All MXene-based mass tags effectively labeled all the 15 immune cell subpopulations identified, with a percentage of positive cells up to 90%. A quantitative evaluation of MXenes at the single-cell level up to 3×10⁷ atoms per cell is also possible by determining the number of atoms per cell from direct atom analysis in biological samples by the dynamic range of atoms by CyTOF solution mode.

In addition, MXene labeling of human PBMCs could be detected by IMC. All 2D materials were successfully identified by IMC, and their signals were mutually exclusive with that of DNA, thus indicating that they did not localize to cell nuclei.

The materials can also be detected as a cocktail of three combined MXene-based mass tags to allow multiplexed labeling of all PBMC subpopulations. In vivo biodistribution using a mixture of MXenes in mice was also possible, revealing MXene accumulation in the liver, blood, spleen, lungs and relative immune cell subtypes. Finally, MXenes can be detectable in intact tissues by MIBI-TOF analysis on mouse organs.

### Additional Disclosure

### A. Immune Profiling and Multiplexed Label-Free Detection of 2D MXenes by Mass Cytometry and High-Dimensional Imaging

There is a critical unmet need to detect and image 2D materials within single cells and tissues while surveying a high degree of information from single samples. Here, we provide a versatile multiplexed label-free single-cell detection strategy based on single-cell mass cytometry by time-of-flight (CyTOF) and ion beam imaging by time-of-flight (MIBI-TOF). This strategy, Label-free sINgle-cell tracking of 2D matErials by mass cytometry and MIBI-TOF Design (LINKED), enables nanomaterial detection and simultaneous measurement of multiple cell and tissue features.

As an example, we selected a set of 2D materials, MXenes, to ensure mass detection within the cytometry range while avoiding overlap with more than 70 currently available tags, each able to survey multiple biological parameters. First, we demonstrated their detection and quantification in 15 primary human immune cell subpopulations. Together with the detection, we used mass cytometry to capture several biological aspects of MXenes, such as their biocompatibility and cytokine production after their uptake. Through enzymatic labeling, we simultaneously evaluated MXenes' mediation of cell-cell interactions. In vivo biodistribution experiments using a mixture of MXenes in mice confirmed the versatility of the disclosed detection strategy and revealed MXene accumulation in the liver, blood, spleen, lungs and relative immune cell subtypes. Finally, we applied MIBI-TOF to detect MXenes in different organs. The label-free detection of 2D materials by mass cytometry at the single-cell level, on multiple cell subpopulations and in multiple organs simultaneously, enables new opportunities in biomedicine.

### 1. Introduction

The ability to track, detect and quantitatively measure nanomaterials in cells and tissues has driven their increasing exploitation in biomedicine. The development of label-free, high-resolution and high-dimensional approaches that simultaneously visualize 2D materials in multiple cell types, thus enabling insight into cell functions and interactions, together with their spatial localization in tissues, will be crucial for translating nanomaterials to clinical applications.

Transition metal carbides, nitrides and carbonitrides (MXenes) are emergent 2D materials with a wide variety of structures and compositions. Although the most studied MXene is Ti₃C₂, more than 30 stoichiometric compositions and at least 20 solid solutions have been reported1. The surfaces of those 2D sheets are covered by functional groups, written as Tx. These groups primarily comprise O, OH and F, and thus are hydrophilic and easily dispersible in water and physiologic media. Because most MXenes have been shown to have biocompatibility and no cytotoxicity, they have been widely explored in diverse fields, including medicine. In particular, simultaneous therapeutic strategies and detection/visualization technologies aimed either at imaging/diagnosis or at guiding therapy have enabled new perspectives in theranostics with 2D materials.

The use of MXenes for photothermal therapy, medical imaging, drug delivery and other biomedical applications. requires the ability to detect MXenes at the tissue and cell levels in vitro and in vivo. An ideal detection modality should enable label-free single-cell level resolution and the simultaneous interrogation of many cellular parameters.

Single-cell mass cytometry by time-of-flight (CyTOF) is a well-established technique based on mass spectrometry to detect metal element-tagged probes, thus allowing for parameter discrimination according to their mass/charge ratio (m z), with minimal overlap or signal background. Whereas CyTOF is limited to cells in suspension, such as circulating blood cells or cells dissociated from tissues, imaging mass cytometry (IMC) and multiplexed imaging by time of flight (MIBI-TOF) similarly use elementally labeled probes to measure dozens of molecular parameters at single-cell resolution in situ. These techniques are becoming increasingly prevalent and have greatly advanced diverse fields including immunology, oncology, neuroscience and infectious diseases.

We have recently demonstrated the use of CyTOF for studying the immunocompatibility of graphen. But because carbon is not trackable with mass cytometry, its detection has been enabled through functionalization with AgInS₂ nanocrystals. However, the material functionalization itself can affect the toxicological profile,^{[30,31]} and the cell interaction and uptake capabilities; it can additionally affect the intrinsic features of 2D materials that are exploitable for therapeutic/bioimaging purposes^{[8]}, thus possibly impairing their biomedical effectiveness.

A versatile approach combining label-free detection with high-dimensional data analysis at the single-cell level would greatly enable the exploration of novel 2D materials in biomedicine. Among those materials, MXenes are notable for their chemical versatility and consequently are attractive candidates for a 2D material that can be detected and imaged by CyTOF, IMC and MIBI-TOF.

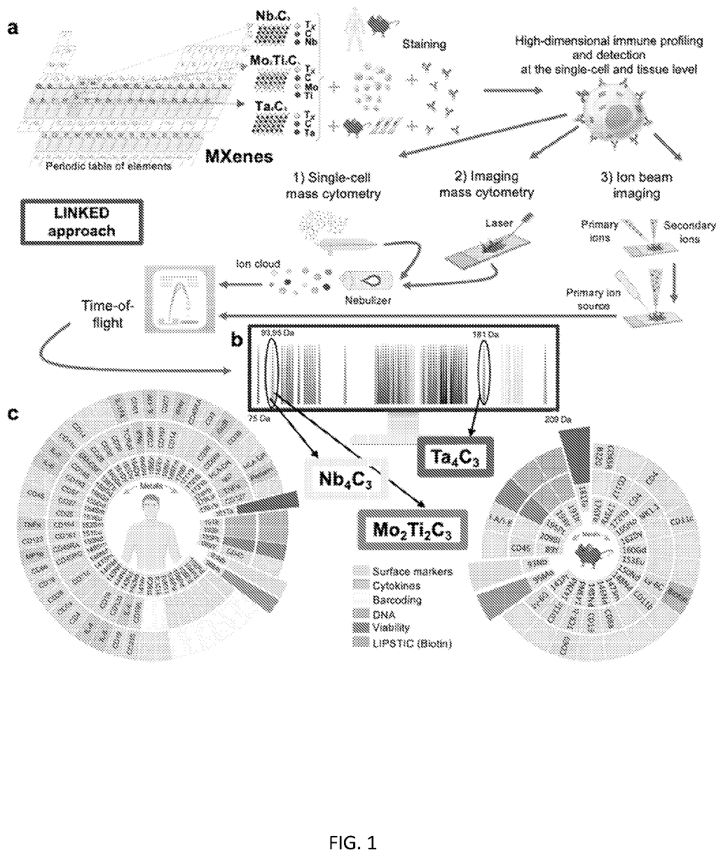

Here, we introduce the Label-free sINgle-cell tracking of 2D matErials by mass cytometry and MIBI-TOF Design (LINKED) approach for the detection of nanomaterials (FIG. 1). In LINKED, we designed 2D materials that can be directly identified by mass cytometry and imaging at the single-cell level (FIG. 1a). This design abrogates the need for additional chemical functionalization, thus overcoming many of the limitations of other imaging approaches.

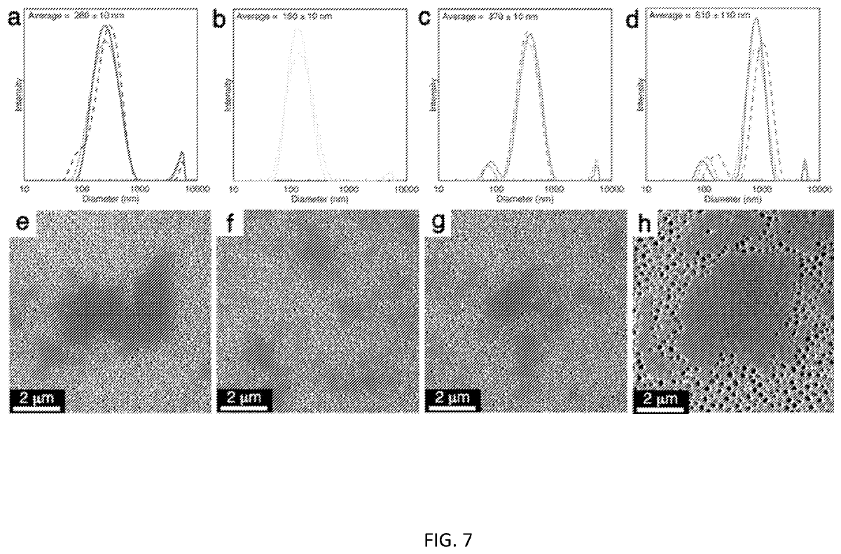

We took several aspects into consideration in designing these MXenes. First, we wanted the materials to fit the detection range of CyTOF and MIBI-TOF, from 79 to 209 in atomic mass. Next, we aimed to avoid any overlap with channels commonly used for metal conjugated antibodies (FIG. 1b). We identified Nb₄C₃, Mo₂Ti₂C₃ and Ta₄C₃ MXenes as eligible candidates, which could be identified in the ⁹³Nb, ⁹⁵Mo and ¹⁸¹Ta channels, respectively. Ti₃C₂, the most widely used MXene for biomedical applications,. was selected as a reference material not visible by mass spectrometry because of its low mass. The full material physicochemical characterization is reported in FIG. 7. We stained peripheral blood mononuclear cells (PBMCs) from healthy human donors, as well as ex vivo and in vivo mouse samples, with a wide variety of metal conjugated-antibodies, then assessed their detection and immune profiling with CyTOF, IMC, and MIBI-TOF. The LINKED driven design of MXenes allowed for the insertion of novel channels, particularly ⁹³Nb and ⁹⁵Mo, which have not previously been explored (FIG. 1b). Moreover, according to LINKED, the selected materials are highly compatible and non-overlapping with currently available antibody panels. Here, we demonstrated their compatibility with more than 70 metal-tagged antibodies, as well as palladium-and cadmium-based barcodes used for ex vivo human immune-phenotyping/immune cell functionality analysis and in vivo mice experiments (FIG. 1c).

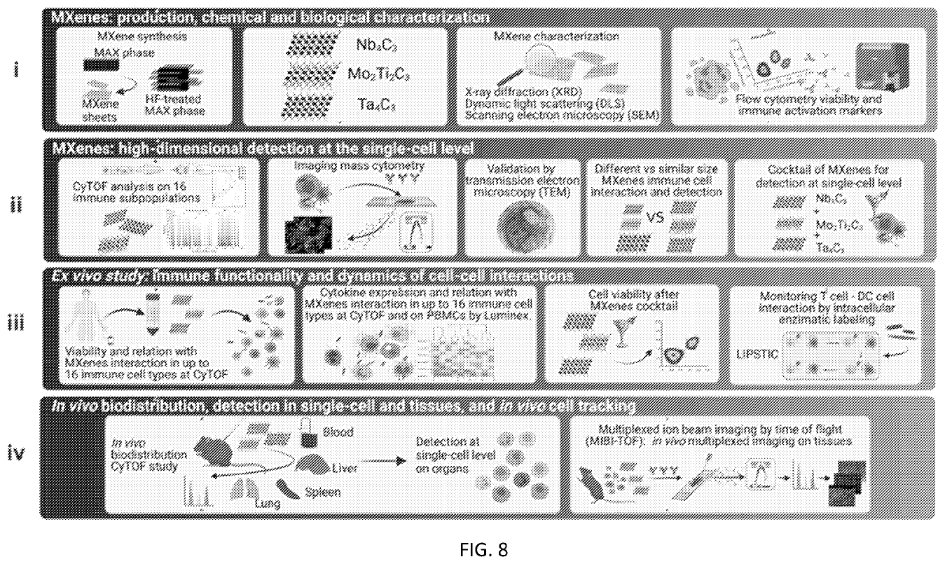

The study workflow is reported in FIG. 8, which shows the various levels of complexity, from material production to their assessment ex vivo in terms of high dimensional immune profiling using different technological platforms, to their detection in multiple human cell types and in several organs in mice. Detection data were then confirmed through MIBI-TOF. In addition, to reveal the possible effects of MXenes on immune cell interactions, we used an enzymatic labeling approach to characterize the complex T cell-dendritic cell interactions, which we adapted and applied to mass cytometry.

### Results and Discussion

MXenes can be detected at the single-cell level without additional chemical functionalization

The tracking of 2D materials is one of their biomedical applications. Therefore, we explored the possibility of detecting MXenes at the single-cell level by CyTOF, simultaneously interrogating many cellular parameters. To this end, we chose the complex pool of PBMCs, consisting of a large variety of immune cell subpopulations, as an ideal ex vivo model for a proof-of-concept study to develop LINKED.

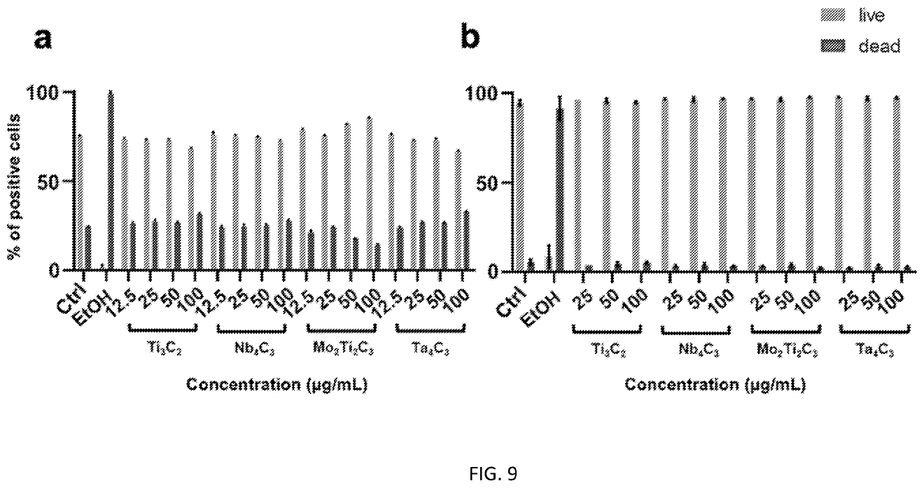

Initially, to investigate the biocompatibility of MXenes, we treated PBMCs with increasing concentrations of Nb₄C₃, Mo₂Ti₂C₃ and Ta₄C₃ for 24 h, then evaluated cell viability with calcein (FIG. 9a) and flow cytometry analysis (FIG. 9b), which showed no cytotoxic effects.

Subsequently, we used CyTOF to explore the potential detection of Nb₄C₃, Mo₂Ti₂C₃ and Ta₄C₃, and reveal the immunological effects on individual cells. The specific atomic masses of the 2D materials selected in this work enabled mass spectrometry detection of Nb₄C₃, Mo₂Ti₂C₃ and Ta₄C₃ in the niobium (⁹³Nb), molybdenum (⁹⁵Mo) and tantalum (¹⁸⁰⁻¹⁸¹Ta) channels, respectively.

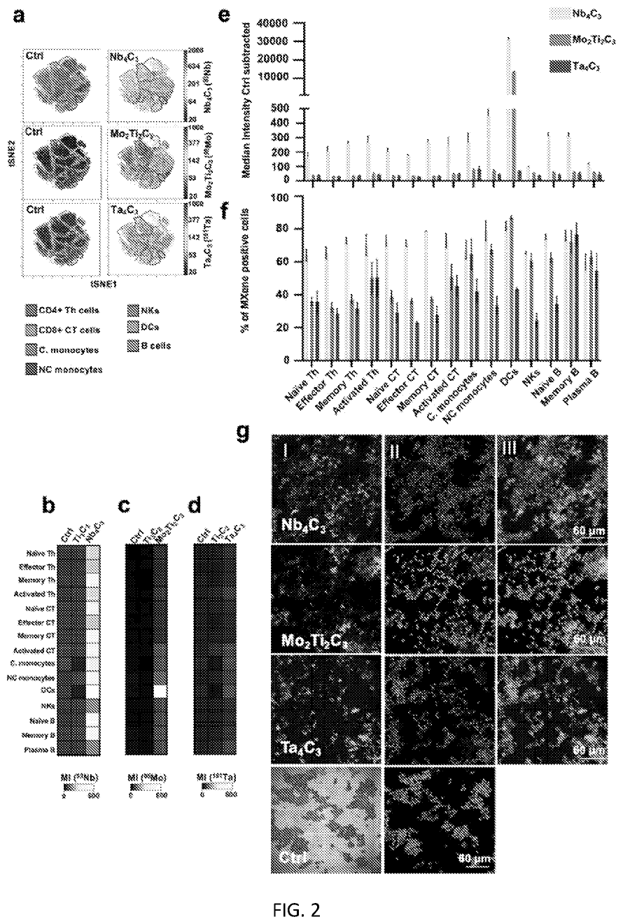

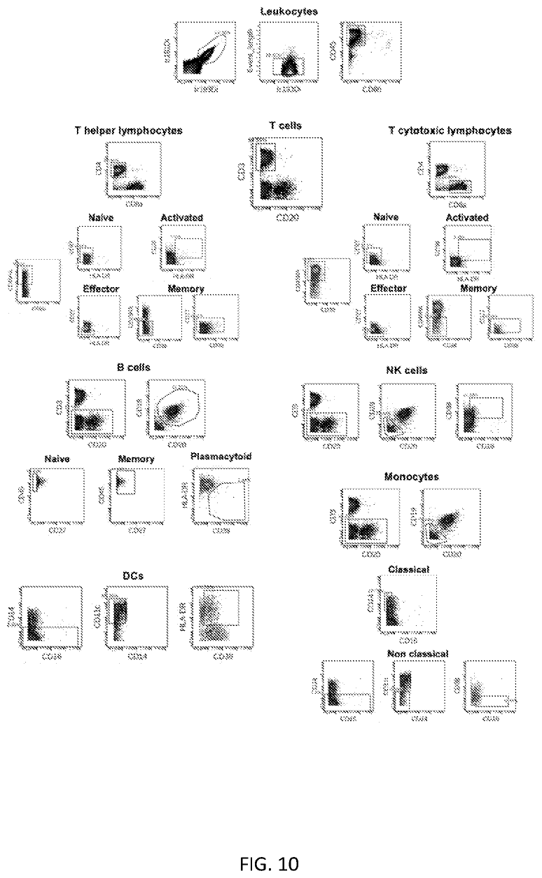

The sub-cytotoxic concentration of 50 μg/mL and the 24 h time-point were chosen on the basis of the results of the viability assays (FIG. 9) and to ensure noticeable changes in immune cell functionality, according to previous experience. Using a panel of 61 metal-tagged antibodies (Table 1), we identified 15 distinct immune cell types according to the expression profiles of several cluster of differentiation (CD) markers present on the cell surfaces (gating strategy in FIG. 10). Computational tSNE analysis was performed as previously reported. The tSNE plots (FIG. 2a), heat maps (FIG. 2b-d) and bar graphs (FIG. 2e,f) indicated that all 2D materials were naturally visible at the single-cell level and interacted with a vast number of immune cell subpopulations.

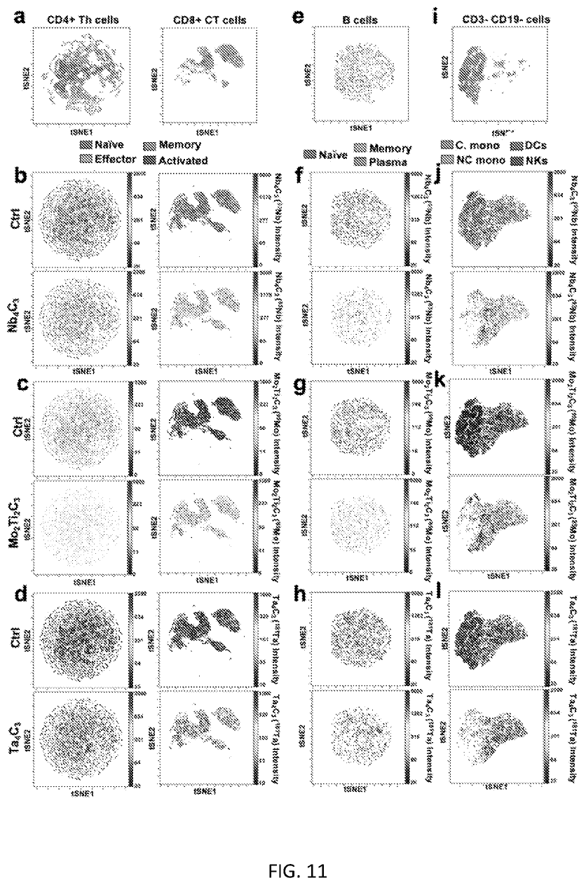

Among the MXenes, Nb₄C₃ had the strongest signal and showed extensive interaction with all immune cell subpopulations identified. In particular, dendritic cells (DCs) showed the most prominent binding to Nb₄C₃. Mo₂Ti₂C₃ and Ta₄C₃ showed similar single-cell signal levels and were similarly trackable in many immune subsets. However, compared with Ta₄C₃, Mo₂Ti₂C₃ had an overall greater ability to interact with the cells, particularly DCs (FIG. 2). The single-cell view by t-SNE confirmed that MXenes interacted with all immune subpopulations (FIG. 11)

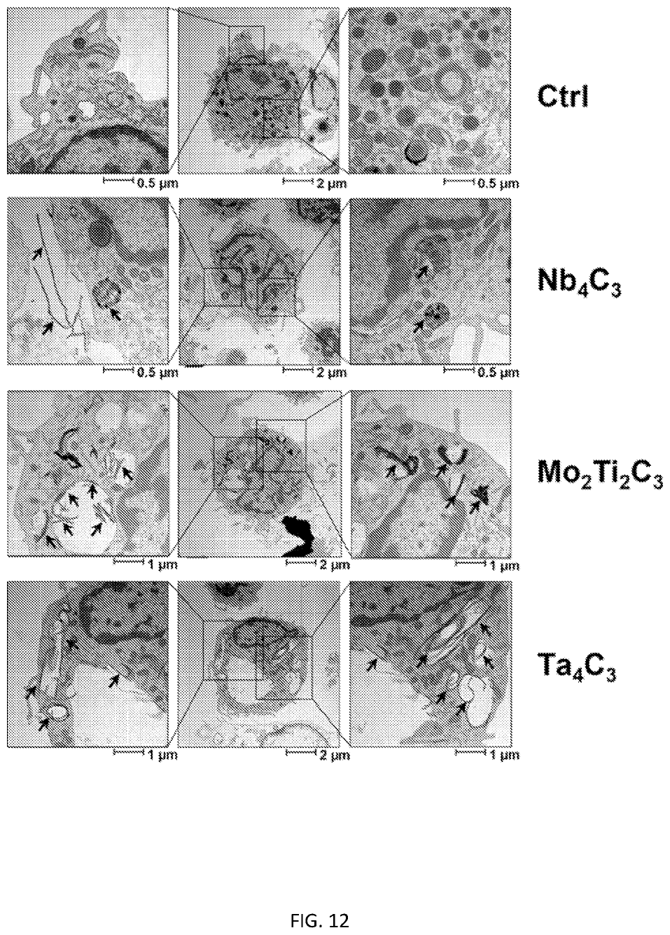

In addition, we evaluated MXene interaction and detection by IMC on human PBMCs (FIG. 2g). All 2D materials were successfully identified by IMC, and their signals were mutually exclusive with that of DNA, thus indicating that they did not localize to cell nuclei. To support the cellular interaction data of CyTOF and IMC, we performed conventional transmission electron microscopy (TEM) analysis of PBMCs. All materials were readily internalized without any ultrastructural signs of cell death (FIG. 12) and were found either as tightly packed ‘bundles’ in the cytoplasm or within large cytoplasmic vacuoles, whereas no materials were found in the cell nucleus, thereby confirming the results obtained with IMC.

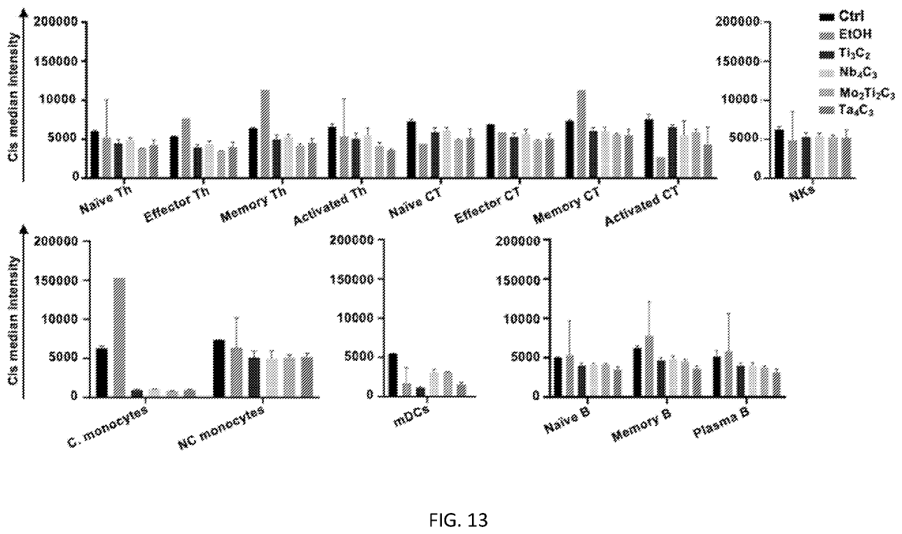

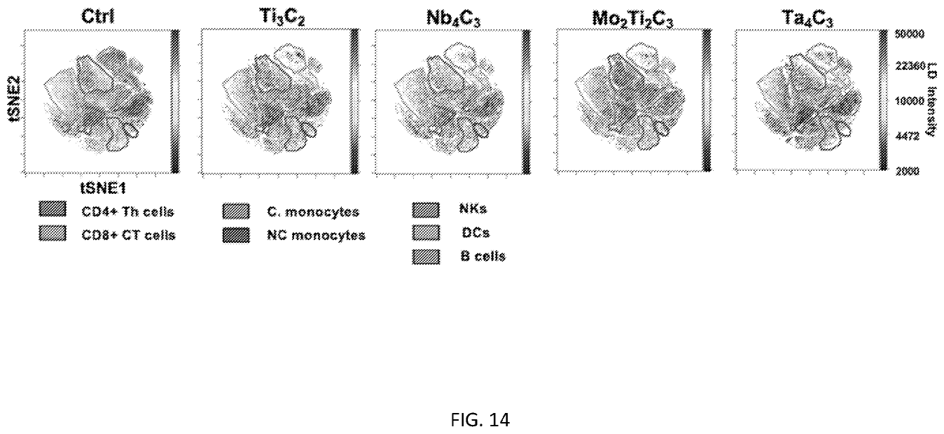

Finally, cisplatin staining revealed that the different 2D material interactions observed among and within the treated cell subpopulations were not associated with a loss of cell viability (FIG. 13 and FIG. 14).

Together, the disclosed results indicated that MXenes are biocompatible in all PBMC subpopulations and can be detected by CyTOF and IMC without chemical functionalization, in conjunction with all conventionally used metal-labeled antibodies.

### MXenes Do Not Affect Immune Cell Functionality

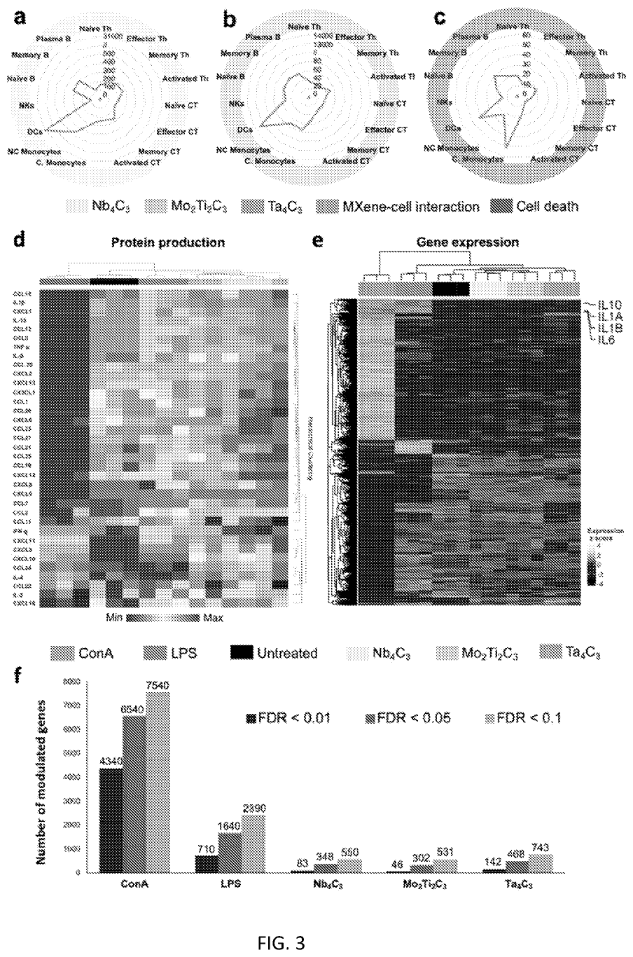

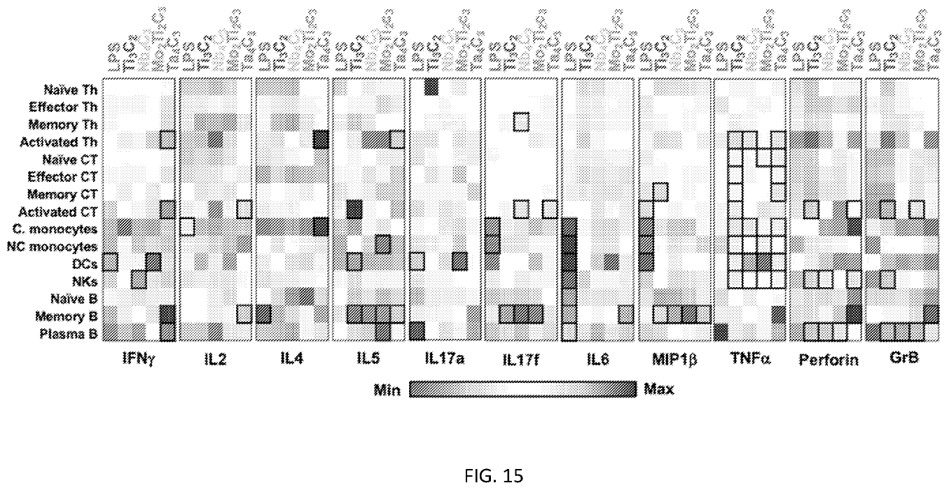

Cytokine production influences several aspects of human health, and their potential modulation by MXenes might be used to guide specific clinical translation applications. Therefore, we assessed immune cell functionality by measuring several secreted cytokines with Luminex assays on the complex pool of PBMCs (FIG. 3d) and by intracellular staining to reveal the different populations via CyTOF (FIG. 15). Heat maps represent the median expression values of the cytokines and activation markers analyzed in all immune cell subpopulations identified after treatment with MXenes.

Overall, the MXenes selected in this work, compared with other 2D materials such as graphene, exhibited only a slight modulation of the inflammatory mediators analyzed. In detail, MXenes induced an overall neutral or inhibitory effect, which was particularly relevant in memory B cells. In contrast, an upregulatory effect limited to several cytokines was observed in DCs and natural killer cells for Ti₃C₂ and Nb₄C₃, respectively, and in non-classical monocytes and activated cytotoxic T cells for Mo₂Ti₂C₃ (FIG. 15).

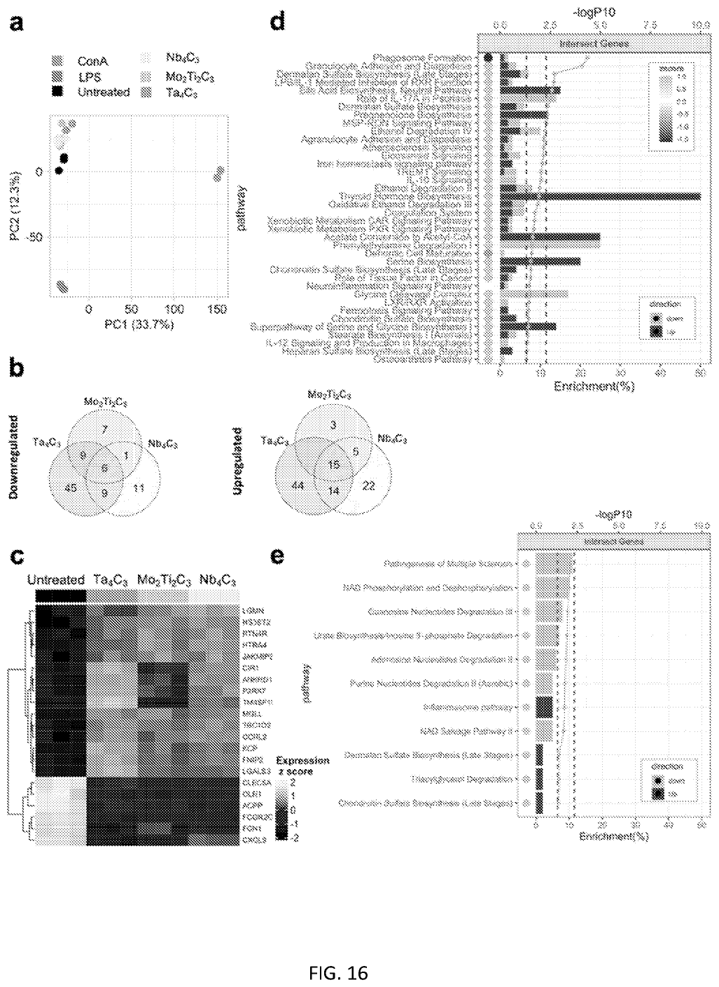

Next, we investigated the effects of the MXenes on PBMC by using mRNA sequencing, a sensitive and unbiased approach (FIGS. 3e and 16). By performing principal component analysis on all analyzed transcripts (N=19,959), we found that MXene-treated samples clustered close to the controls, and separately from concanavalin A (ConA)- and lipopolysaccharide (LPS)-treated samples, thus indicating that only minimal perturbations were induced by the investigated materials (FIG. 16a). Overall, we did not observe genome-wide changes induced by MXenes, even with permissive p value cut-offs (FIG. 3e,f). All materials showed modest effects on human PBMCs, modulating the expression of fewer genes than the positive controls ConA and LPS (FIG. 3e,f). In particular, even if all materials had similar effects on gene expression modulation, Ta₄C₃ had slightly greater effects than Mo₂Ti₂C₃ and Nb₄C₃. The expression of 142, 46 and 83 and genes was modulated by Ta₄C₃, Mo₂Ti₂C₃ and Nb₄C₃, respectively, whereas the positive controls ConA and LPS modulated the expression of 4,340 and 710 genes, respectively (FDR<0.01) (FIG. 3f). Furthermore, pathway-enrichment analyses on genes coherently modulated by the three MXenes showed only modest enrichment of immune-related pathways without a clear indication of activation or inhibition.

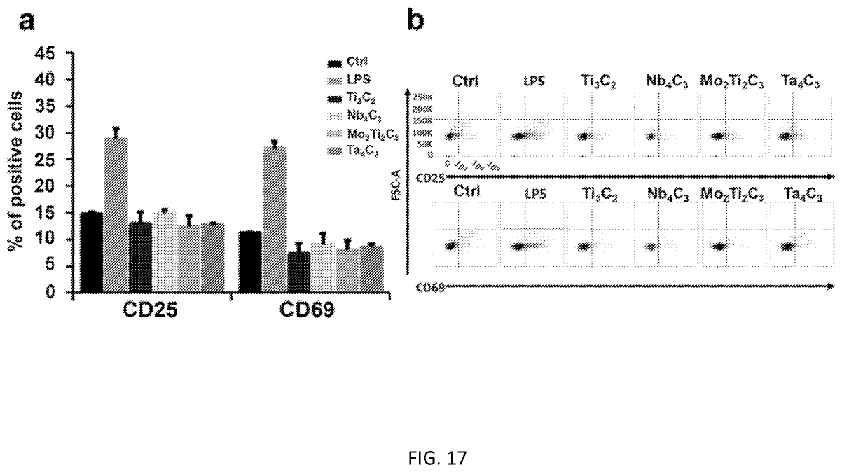

To further investigate the effects of the selected 2D materials on immune cell functionality, we monitored the expression of CD25 (α-chain of the IL-2 receptor) and CD69 (C-type lectin protein), which are late and early activation markers, respectively, by using flow cytometry after treatment with Nb₄C₃, Mo₂Ti₂C₃ or Ta₄C₃, and Ti₃C₂ as an additional control (FIG. 17). No significant increase in the expression of the selected markers was observed, thereby confirming the overall neutral effect of the materials on immune cell function.

Collectively, these results indicated that MXenes show good immunocompatibility.

### MXene Modularity Enables Their Customization for Optimal Cell Viability and Enhanced Cell Internalization

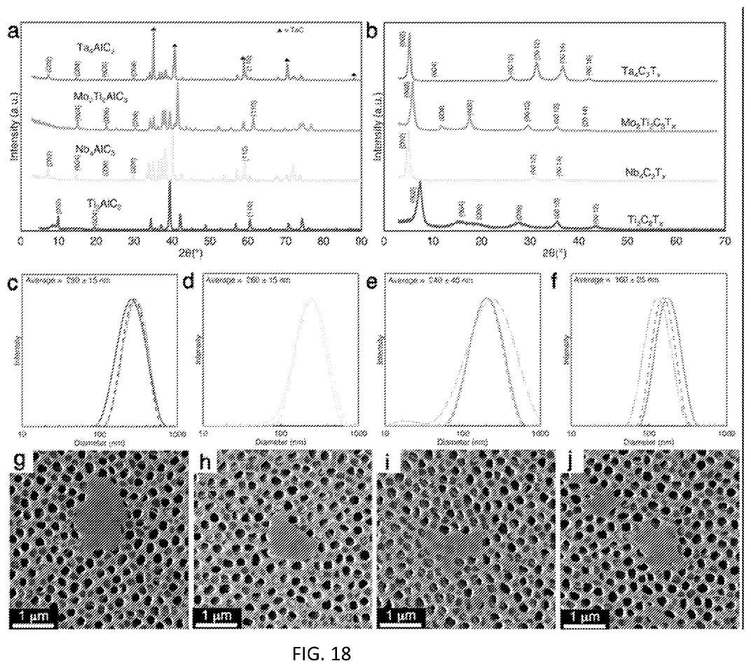

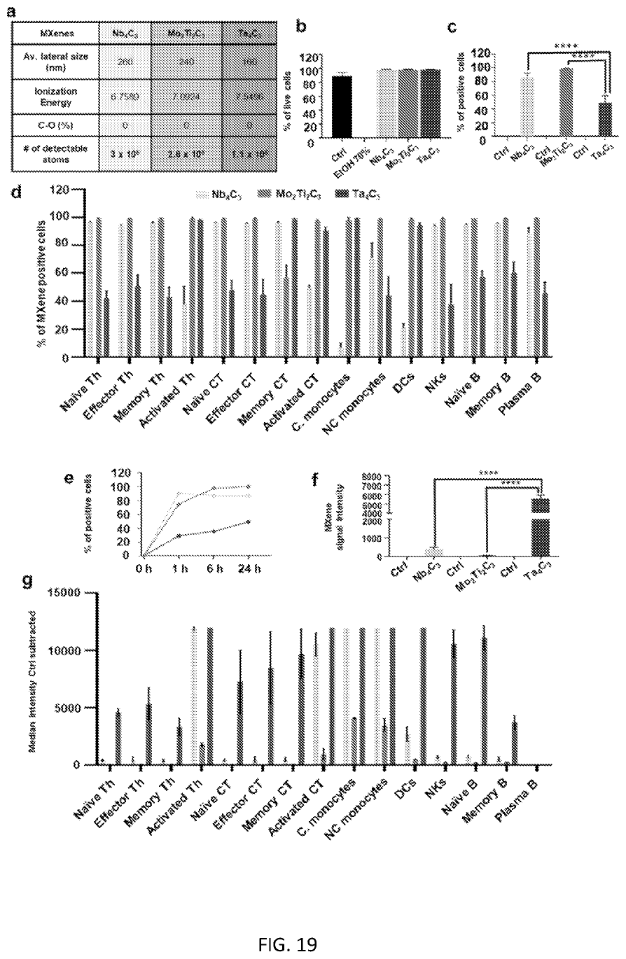

For another 2D material, graphene, the lateral size has been shown to play a role in cell viability and function. Therefore, we investigated the effects of MXene lateral size on their uptake, viability, activation and detection profile. To this end, we produced Nb₄C₃, Mo₂Ti₂C₃ and Ta₄C₃ MXenes with more similar lateral sizes (260, 240 and 160 nm, respectively) (FIG. 18) compared to those in the previous experiments (150, 370 and 810 nm). In FIG. 19a, for each material, we report the CyTOF ionization energy and the number of atoms detectable by mass cytometry. First, the cell viability of MXene-treated PBMCs was analyzed with a cisplatin protocol with CyTOF. We confirmed in FIG. 19b that all materials showed excellent biocompatibility (98.7, 98.8 and 98.7% in live cells for Nb₄C₃, Mo₂Ti₂C₃ and Ta₄C₃, respectively). The percentage of cells positive for Nb₄C₃, Mo₂Ti₂C₃ and Ta₄C₃ ranged between 40% for Ta₄C₃ and >90% for Mo₂Ti₂C₃ and is reported for the total PBMCs (FIG. 19c) and all 15 immune subpopulations identified (FIG. 19d). The results confirmed that all 2D materials were naturally visible at the single-cell level and were able to interact with a vast number of immune cell subpopulations. Moreover, we demonstrated how the modulation of MXene lateral size by design can increase their detection signal and uptake. In addition, the time course analysis of MXene cell uptake after treatment of PBMCs for 1, 6 and 24 h demonstrated that even after a short exposure time (1 h), MXenes were internalized by immune cells (FIG. 19e). The median intensity of Nb₄C₃, Mo₂Ti₂C₃ and Ta₄C₃ signals after 24 h is also reported as bar plots for total PBMCs (FIG. 19f) and all cell subpopulations (FIG. 19g).

### MXene Cocktail Safety Profile Indicates No Effects on Cell Internalization Capability

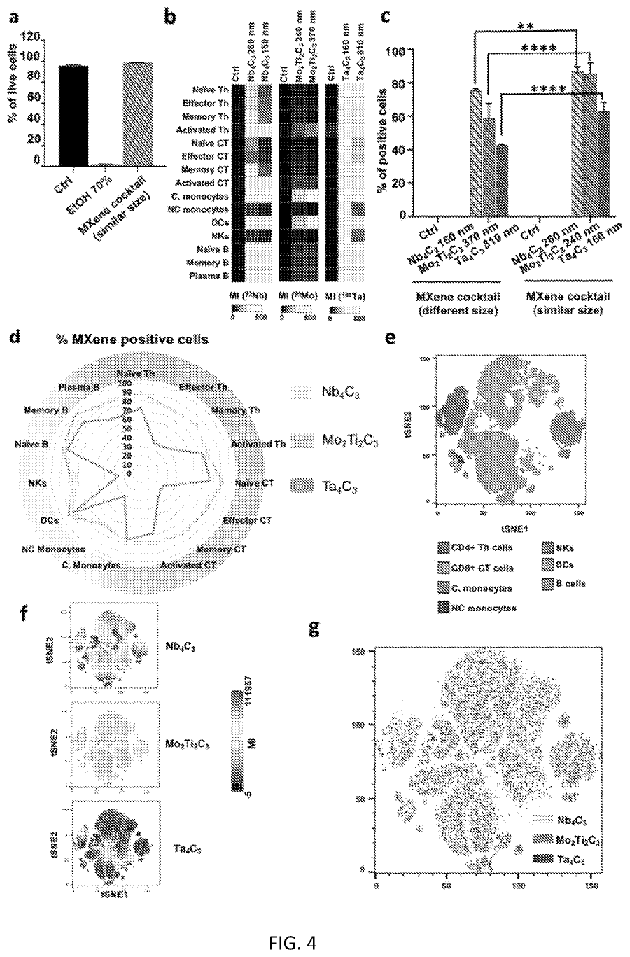

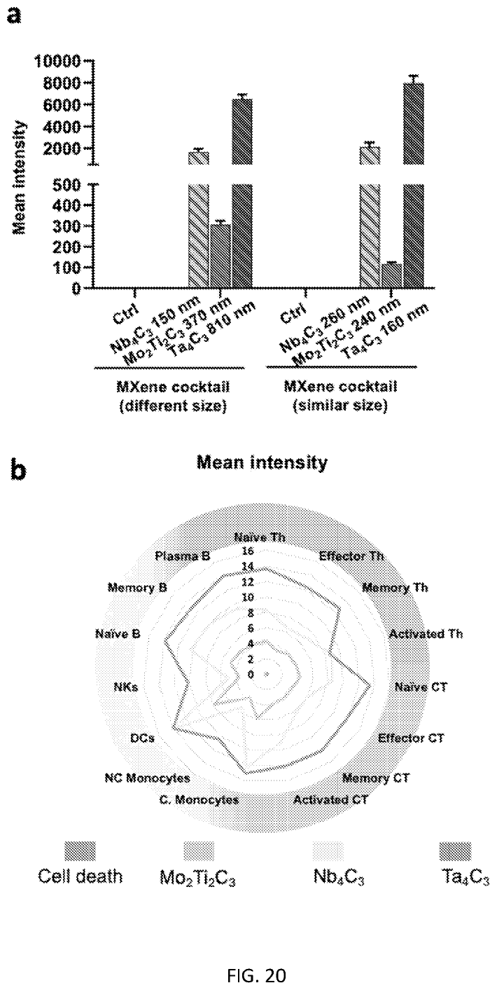

Next, we evaluated whether MXenes might be combined to allow multiplexed detection of the materials. To this end, we incubated PBMCs with 150 μg/mL of a cocktail of the three MXenes with similar lateral size: Nb₄C₃, Mo₂Ti₂C₃ and Ta₄C₃ (50 μg/mL each for 24 h); we then stained with 61-plex panel markers and assessed viability (Table 1). We observed high biocompatibility of the materials even as a cocktail and at high concentrations (99.9% cell viability at 150 μg/mL) (FIG. 4a). The heat maps show the MXene detection at the single-cell level and their ability to interact with 15 immune cell subsets without affecting their viability (FIG. 4b). Moreover, according to bar plots representing the mean intensity (FIG. 20a) and the percentage of positive cells to MXenes (FIG. 4c) in total PBMCs after treatment for 24 h with the cocktail of MXenes with different sizes (Nb₄C₃—150 nm, Mo₂Ti₂C₃—370 nm and Ta₄C₃—810 nm, 50 μg/mL each) or similar sizes (Nb₄C₃—260 nm, Mo₂Ti₂C₃—240 nm and Ta₄C₃—160 nm, 50 μg/mL each), the differences in lateral size were found to modulate the binding, the uptake and consequently the detection of the materials with the cells, even when the MXenes were administered as a cocktail. The MXene cocktail with similar lateral sizes showed interaction with all immune cell subpopulations, as indicated by the percentage of MXene positive cells (FIG. 4d), and a high biocompatibility regardless of the extent of interaction (FIG. 20b). In particular, Nb₄C₃ and Mo₂Ti₂C₃. despite their lower mean signal intensity (MI) than that of Ta₄C₃ (FIG. 4b), showed a similar higher percentage of single-cell positive cells, which were trackable in many immune subsets, whereas Ta₄C₃ was more selective for specific immune cell subpopulations (FIG. 4d). tSNE analysis reporting all identified major immune subpopulations, MXene individual mean signal intensity, and a tSNE overlay representation of MXene MI in immune cell subpopulations are shown in FIGS. 4e, 4f and 4g, respectively.

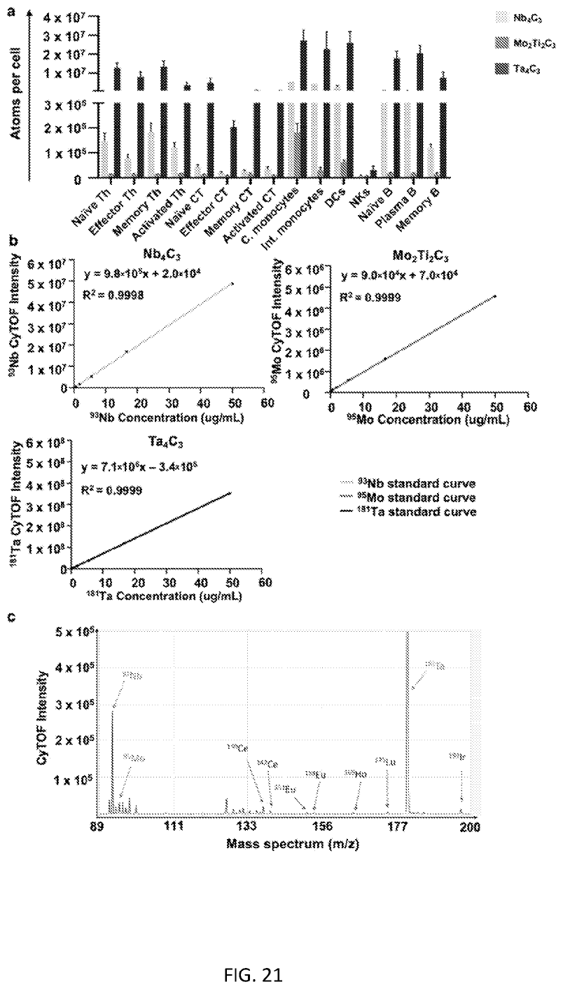

We next performed a quantitative evaluation of MXenes at the single-cell level. To this end, we treated PBMCs with a cocktail of Nb₄C₃—260 nm, Mo₂Ti₂C₃—240 nm and Ta₄C₃—160 nm, and determined the number of ⁹³Nb, ⁹⁵Mo and ¹⁸¹Ta atoms per cell from direct atom analysis in all PBMC subpopulations (FIG. 21a). In every cell type, a greater number of atoms per cell was observed for Ta₄C₃, followed by Nb₄C₃ and Mo₂Ti₂C₃.

The dynamic range of ⁹³Nb, ⁹⁵Mo and ¹⁸¹Ta, and the mass spectra of Nb₄C₃, Mo₂Ti₂C₃ and Ta₄C₃ determined with CyTOF solution mode are also reported in FIGS. 21b and 15c, respectively. The CyTOF signal intensities are plotted against concentrations of the materials (FIG. 21b). The dynamic range of quantification of the materials was linear up to 50 μg/mL. At the same concentrations, a difference of one or two orders of magnitude was observed in Mo₂Ti₂C₃ intensity compared with Nb₄C₃ and Ta₄C₃ intensity, respectively.

### MXenes Do Not Alter Dendritic Cell Interactions with T Cells

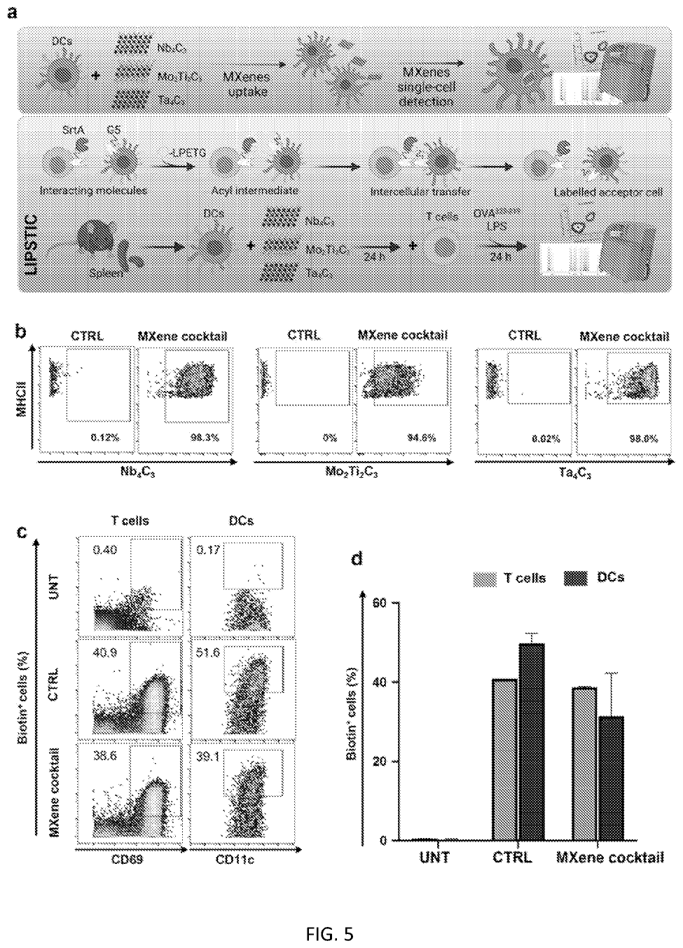

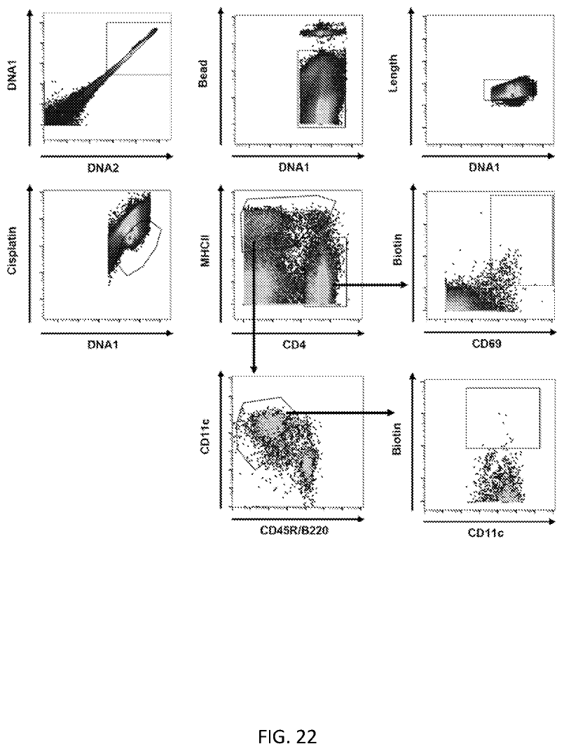

Because Mxenes showed notable interactions with immune cells and dendritic cells, we investigated the effects on the interaction between antigen presenting DCs and T cells—an essential phenomenon underlying the activation of the immune response. To this end, we exploited the LIPSTIC approach and coupled it with the CyTOF strategy for the analysis of cell-cell interactions after MXene treatment of DCs (FIGS. 5a and 22). This coupled approach has not previously been reported. We isolated DCs and naïve CD4⁺ T cells from Cd40^{G5/G5} and Cd40lg^{SrtA/Y} CD4−Cre⁺ OT-II mice, respectively, so that the DCs constitutively expressed G5-CD40 protein, whereas the CD4⁺ T cells expressed CD40L-SrtA as well as a transgenic TCR (OT-II) that specifically recognized a chicken ovalbumin derived epitope (OVA₃₂₃₋₃₃₉ or OT-II peptide). According to previous findings, CD4⁺ T cells present CD40L-SrtA on their membranes after cognate interaction with DCs. If a SrtA substrate (e.g., biotinylated LPETG) is provided, the enzyme transiently binds the substrate via formation of an acyl intermediate, then mediates its transfer to nearby DCs expressing G5-CD40. In brief, this strategy allows for the identification and tracking of cells that undergo interactions, which are visualized as biotin⁺ populations. To assess whether MXene internalization by DCs might affect the above-mentioned process, we treated DCs from LIPSTIC model mouse spleens with the MXene cocktail for 24 h. At the end of the incubation, treated cells were washed and maintained in culture for an additional 24 h, in the presence of untreated T cells, OT-II peptide (9.25 μg/mL) and LPS (10 μg/mL) to allow for antigen presentation. The biotinylated SrtA substrate (biotin-LPETG) was provided during the final 30 minutes of incubation. CyTOF analysis was used to monitor MXene uptake by DCs (owing to the nanomaterials' detectable masses and compatibility with the antibody panel reported in Table 2) as well as substrate transfer between T cells and DCs (FIG. 5a). As shown in FIG. 5b, all materials were efficiently taken up by DCs (>90% MXene positive cells). The representative dot plots shown in FIG. 5c demonstrated that LIPSTIC labeling in CD4⁺ T cells (left) and DCs (right) was not impaired by treatment with the cocktail of Mxenes. Indeed, no significant difference in the percentage of biotin⁺ DCs and CD4⁺ T cells was observed when Mxenes were provided, as highlighted by the grouped bar plots in FIG. 5d. Overall, these results indicated that intercellular communication between DCs and T cells was preserved even after MXene uptake by DCs.

### MXenes are Detectable after In Vivo Administration

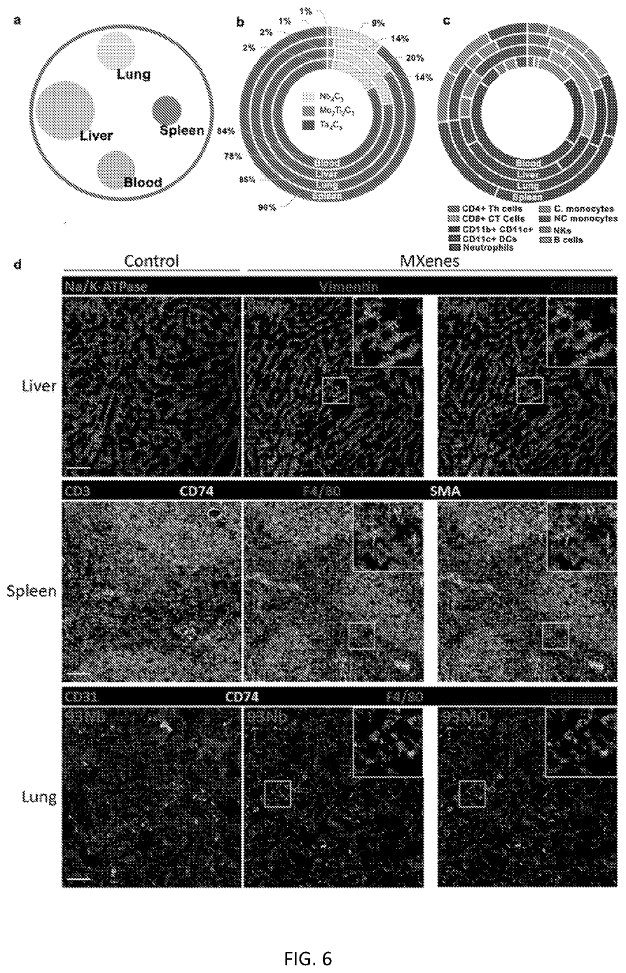

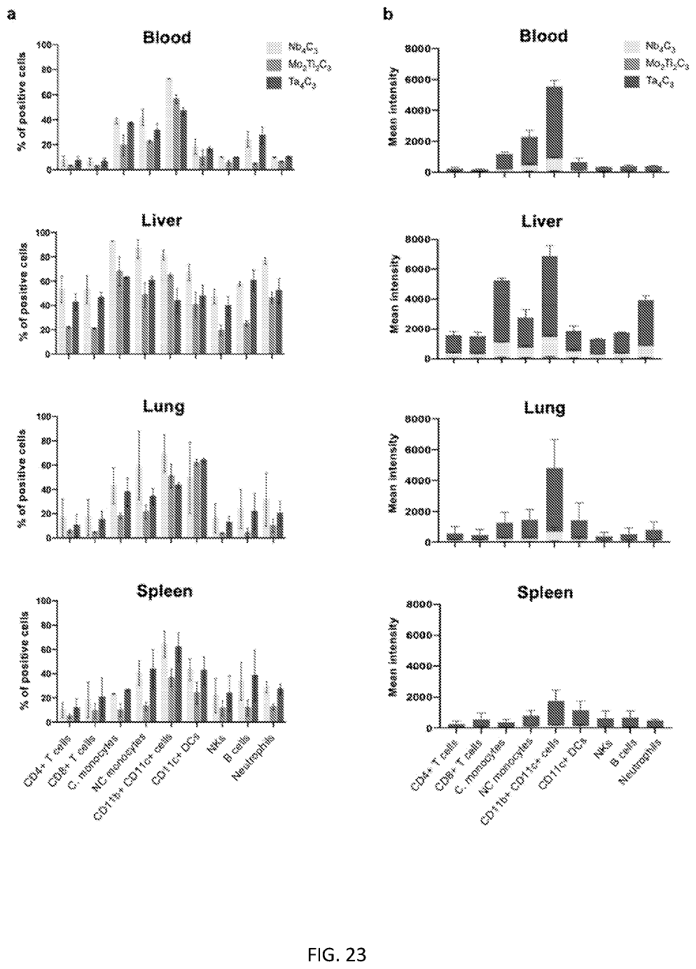

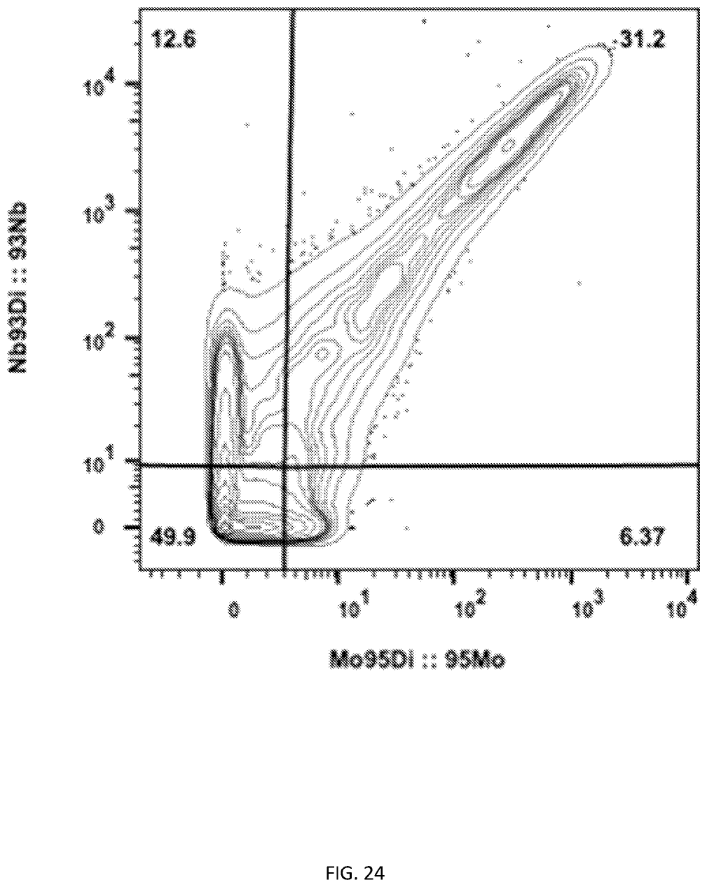

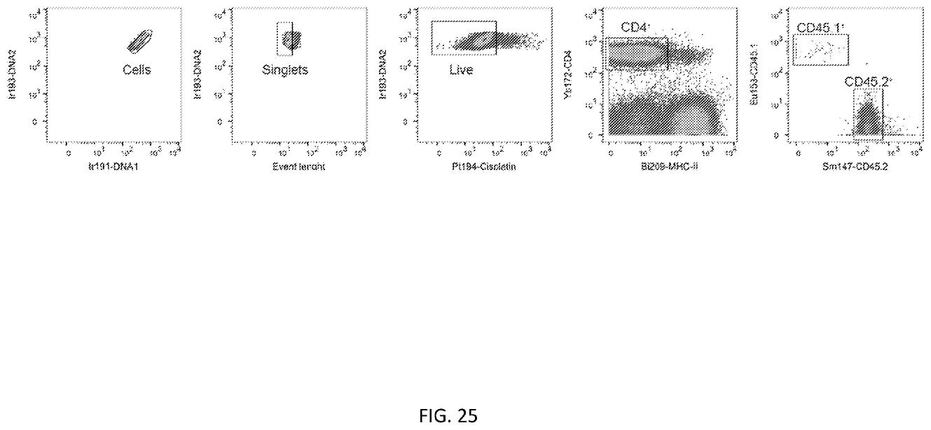

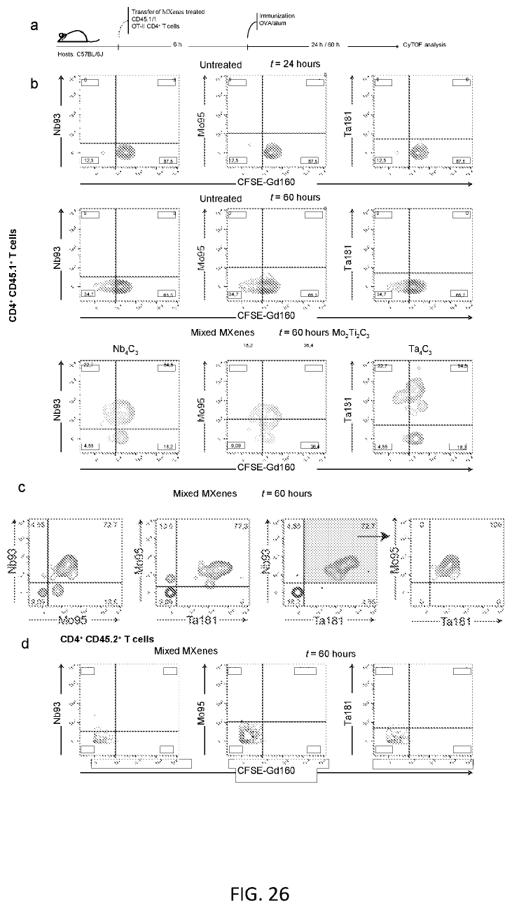

We then investigated the in vivo biodistribution and detection of the MXene cocktail at the tissue and single-cell level by CyTOF and MIBI-TOF. To this end, we I.V. injected C57BL/6J male mice with 20 mg/kg of a cocktail of MXenes (Nb₄C₃, Mo₂Ti₂C₃ and Ta₄C₃) for 24 h (FIG. 6). The liver, lung, spleen and blood were subsequently analyzed, and all materials were well detectable at the tissue level and by multiple single-cell staining (3). The liver had the highest MXene cocktail signal intensity, followed by the blood, lung and spleen (FIG. 6a). In detail, Ta₄C₃ showed the highest signal, followed by Nb₄C₃ and Mo₂Ti₂C₃, as revealed by the signal intensity deconvolution for each MXene in all organs analyzed (FIG. 6b). The MXene MI signal intensity detected in each immune cell subpopulation analyzed per organ revealed that MXenes were detectable in all cell subsets analyzed, particularly in CD11b⁺ and CD11c⁺ dendritic cells (FIG. 6c). Grouped bar plots showed the percentage of positive cells (FIG. 23a) and quantified MXene MI (FIG. 23b) for all immune cell subpopulations identified per organ. A representative contour plot of gated CD45⁺ cells, isolated from the livers of MXene-treated mice, is shown in FIG. 24.

Finally, to test whether MXenes might be detectable in intact tissues, we performed MIBI-TOF analysis on mouse organs (FIG. 6d). We stained the tissues with a panel of nine metal-labeled antibodies, as detailed in 4, and visualized them by MIBI-TOF, according to previously reported protocols for tissue preparation. A specific MXene signal was detected in all tissues, at both the niobium (⁹³Nb) and the molybdenum (⁹⁵Mo) channels, alongside the expected antibody staining (4), with no signs of tissue damage. The signal in the niobium channel was brighter, exhibiting higher sensitivity of detection of the presence of material. In particular, MXenes were detected mainly in the liver and spleen, followed by the lungs. The materials colocalized and also showed intracellular staining of non-immune cells, mainly in the liver.

Thus, we successfully identified the materials in intact mouse tissues, together with multiple antibody staining. These findings provide a 2D material detection solution for the large community of scientists performing in vivo pre-clinical studies.

This study indicated that LINKED can be readily applied to a wide variety of 2D materials to provide new insights into their toxicological profile and translate them into clinical systems, because the approach: i) tracks 2D materials at the single-cell level, ii) represents a new frontier in 2D material biocompatibility assessment, iii) provides a versatile high-dimensional strategy to investigate 2D material functional interactions with a wide variety of cell populations simultaneously (exemplified here by human immune cells) and iv) promotes the development of new nano-based theranostic applications. LINKED's 2D material detection design should substantially advance the state of the art and accelerate new advances and discoveries in nanotechnology and medicine.

### Observations

We presented evidence of label-free single-cell mass cytometry detection of 2D materials, i.e., MXenes containing specific transition metals. The 2D materials were reliably detected by imaging mass cytometry and multiplexed ion beam imaging MIBI-TOF, thus paving the way for their detection in a wide variety of organs for a myriad of biomedical applications. This study provides the first demonstration of imaging of MXenes by using three different mass cytometry techniques in combination with 76 mass cytometry probes (antibodies). Moreover, we used the ⁹³Nb and ⁹⁵Mo channels, which have not previously been explored for commercial mass cytometry tags. We tested the MXenes simultaneously on 15primary human immune cell subpopulations and successfully detected material uptake and cell interactions by CyTOF and imaging mass cytometry. Simultaneously, we demonstrated their excellent biocompatibility through high-dimensional immune and functional profiling by CyTOF. By using TEM, flow cytometry, Luminex assays and RNA-seq, we demonstrated the bio-and immunocompatibility of MXenes in terms of cytokine and chemokine production, expression of activation markers and effects on whole-genome expression. We found that the modulation of MXenes' lateral size can be used to increase their detection signal and uptake. We also demonstrated the possibility of detection and quantification of MXenes from a multi-material mixture: a MXene cocktail. By adapting the innovative LIPSTIC approach, based on enzymatic labeling, for CyTOF, we showed that MXenes do not interfere with the interaction of two key players in the immune response: T cells and DCs. CyTOF on mice revealed the biodistribution of the MXene cocktail in vivo at the tissue and single-cell level, indicating MXene imaging properties in multiple organs while capturing immune cell internalization. Finally, we used multiplex ion beam imaging, which has not previously been used for nanomaterial detection, to detect the presence of MXenes in different organs, and observed no signs of tissue damage.

The possibility of detecting MXenes by multiple mass cytometry platforms at the single-cell and tissue levels is expected to tremendously advance preclinical research on this large and highly promising family of 2D materials. The disclosed strategy is not subject to the classical limits of ordinary labeling methods or imaging approaches, and it has the potential to be further developed as a technological pipeline that could be extended to other classes of 2D materials with similar chemical properties to those tested in this study.

### Experimental Section/Methods

### Preparation and Characterization of MXenes

### MAX Precursor Synthesis

To synthesize the MAX precursors, Ta (−325 mesh, Alfa Aesar, 99.97%), TiC (<2 μm, Alfa Aesar, 99.5%), Nb (−325 mesh, Beantown Chemicals, 99.99%), Mo (−250mesh, Alfa Aesar, 99.9%), Ti (−325 mesh, Alfa Aesar, 99.5%), Al (−325 mesh, Alfa Aesar, 99.5%), and graphite (−325 mesh, Alfa Aesar, 99%) powders were used. To produce Ti₃AlC₂, a 2:1:1 atomic ratio of TiC:Ti:Al (50 g total) was mixed. For Nb₄AlC₃, a 4:1.1:2.7 atomic ratio (10 g total) of Nb:Al:C was used. For Mo₂Ti₂AlC₃, a 2:2:1.3:2.7 atomic ratio (10 g total) of Mo:Ti:Al:C was mixed. And for Ta₄AlC₃, a 4:1:3 atomic ratio (7 g total) of Ta:Al:C was utilized. The powder mixtures were then mixed in a 2:1 ball:powder ratio with 5 mm alumina balls. The mixtures were ball milled at 60 rpm for 24 h prior to high temperature annealing.

All high-temperature annealing reactions were conducted in a Carbolite furnace, with heating and cooling rate of 3° C., and 200 cm³ min⁻¹ flow of ultra-high purity Ar (99.999%). For Ti₃AlC₂, the mixture was heated to 1400° C. for 2 h. For Nb₄AlC₃, the powders were heated to 1650° C. for 4 h. The Mo₂Ti₂AlC₃ powder mixture was heated to 1600° C. for 4 h. Finally, to produce Ta₄AlC₃, the mixture was heated to 1400° C. for 8 h. After cooling, the porous compacts were milled using a TiN-coated milling bit and sieved through a 400-mesh sieve, producing powders with a particle size <38 μm. All experiments on this study were conducted on a single batch of MAX to eliminate any artifacts from variation between MAX synthesis batches.

### MXene Synthesis

To topochemically synthesize (selective Al etching) the MAX phases to produce MXenes, HF (Acros Organics, 48-50 wt. %; 29 M), HCl (Fisher Scientific, 37 wt. %; 12 M), and deionized (DI) water (15 MΩ resistivity) was utilized. For all reactions, Teflon coated stir bars were used with a stirring rate of 300 rpm. Ti₃C₂Tₓ was produced by etching 1 g of Ti₃AlC₂ in a 1:3:6 volumetric ratio (20 mL total) of HF:H₂O:HCl for 24 h at 35° C. To synthesize Mo₂Ti₂C₃Tₓ, 1 g of Mo₂Ti₂AlC₃ was added to 20 mL of 48-50 wt. % HF and stirred for 96 h at 55° C. Nb₄C₃Tₓ was synthesized by adding 1 g of Nb₄AlC₃ to 20 mL of 48-50 wt. % HF and stirred for 120 h 35° C. Ta₄C₃Tₓ was synthesized from 1 g Ta₄AlC₃ in 20 mL of 48-50 wt. % HF for 72 h at 35° C. Tr represents the surface functional groups (═O, —OH, —F) on the MXene surface after etching and is omitted afterwards for brevity. After the appropriate etching time, the mixtures were washed with DI water by centrifugation. The post-reacted mixtures were mixed with 150 mL DI H₂O, then were centrifuged at 3,500 rpm for 10 min. The acidic supernatant was decanted, with the multilayer MXene remaining as the sediment. New DI H₂O was added, with the sediment redispersed. This process was repeated eight times. This ensured that the sample was fully neutral, and any excess adsorbed acid was removed.

All multilayer MXenes in this study were delaminated in 20 mL of a 5 wt. % TMAOH solution (TMAOH; Sigma Aldrich, 25 wt. % in H₂O). The MXenes were stirred for 12 h at 300 rpm at 35° C. After stirring, the samples were placed into 50 mL centrifugation tubes. DI water was added, and the samples were centrifuged at 10,000 rpm for 20 min. The supernatant was decanted off, and the MXene was redispersed in fresh DI water. This procedure was repeated five times to ensure that all excess TMAOH was removed. Following this last cycle, the MXene was redispersed in 50 mL DI water, then was centrifuged at 3,500 rpm for 10 minutes. The supernatant was collected, then centrifuged again at 3,500 rpm for 10 min, the supernatant was carefully decanted for use. This ensured that only single-flake MXene remained in the solution.

A small fraction of this solution was then vacuum filtered on Celgard membranes (64 nm pore size, 3501 coated polypropylene), which were first washed with ethanol, then deionized water, to produce free-standing films for X-ray diffraction (XRD). By measuring the weight of the produced films (and considering the solution volume added), the concentration of the films was determined.

### MXene Characterization

XRD patterns of the powders and films were collected on a Rigaku Smartlab (40 kV and 30 mA) diffractometer using Cu Kα radiation. The conditions were as follows: (i) for the MAX powder, step scan 0.02, 3-90 (2θ), step time of 1 s; (ii) for the MXene films, a step scan of 0.03, 3-70 (2θ), step time of 0.5 s was used. Scanning electron microscopy (SEM) was conducted on a dual-beam focused ion beam (Strata DB235, FEI). The MXene flakes were drop-cast onto a porous alumina substrate. Pt was deposited onto the flake and substrate to minimize charging. DLS (Zetasizer Nano ZS, Malvern Instruments) was performed to analyze the size of the MXene flakes. Three measurements were taken of each sample and the average value was reported. Characterization of the MXenes is reported in FIG. 7. All materials were tested for possible contaminations.

### Peripheral Blood Mononuclear Cells (PBMCs)

PBMCs were harvested from ethylenediamine tetraacetic acid (EDTA)-venous blood from informed healthy donors (25-50 years old) using a Ficoll-Paque (GE Healthcare, CA, USA) standard separation protocol. Informed signed consent was obtained from all the donors. Cell separation and experiments were performed immediately after blood drawing. PBMCs were cultured in 24-well plates in RPMI 1640 medium (Life Technologies), supplemented with 1% penicillin/streptomycin (Life Technologies), and 10% heat-inactivated fetal bovine serum (Life Technologies). At least 1×10⁶ cells/sample in each experiment were used. Experiments were carried out using multiple healthy donors and technical triplicate.

### Calcein AM/Ethidium Homodimer-1 Assay

PBMCs were treated for 24 h with different concentrations of (12.5, 25, 50 and 100 μg/mL) of each material and calcein AM/ethidium homodimer-1 staining was performed by incubating cells with 2 μmol/L calcein AM and 5 μmol/L ethidium homodimer (Live/Dead® Viability/Cytotoxicity kit, Invitrogen) for 45 min at 37° C. in the dark. Ethanol 70% was used as a positive control, while samples incubated with medium alone were used as negative controls. The assay discriminates live from dead cells by simultaneously staining with green-fluorescent calcein-AM (excitation wavelength of 485 nm and emission wavelength of 530 nm) to indicate intracellular esterase activity and red-fluorescent ethidium homodimer-1 (excitation wavelength of 530 nm and emission wavelength of 645 nm) to indicate loss of plasma membrane integrity. Plasma membrane integrity and esterase activity were measured by a Fluorescence Microplate Reader (TECAN infinite M200PRO, Switzerland).

### Cytotoxicity and Cell Activation by Flow Cytometry

PBMCs were treated with increasing concentrations of each material (i.e., 25, 50 and 100 μg/mL) for 24 h and Fixable Viability Stain 780 (FVS780, BD Horizon™) was used to discriminate viable from non-viable cells. Staining was performed in the dark for 30 min. Ethanol at 70% was used as a positive control, while samples incubated with medium alone were used as negative controls. Cells were processed by flow cytometry (LSR Fortessa X-20, BD Bioscience, CA, USA), while data were analyzed by FlowJo™ Software.

In addition, PBMC activation was analyzed after treatment with each material (50 μg/mL) for 24 h. Cells were stained to identify immune activation markers. CD25 and CD69 (PE-conjugated anti-CD25, M-A251 clone; FITC-conjugated anti-CD69, FN50 clone; BD Bioscience, CA, USA) were used as activation markers. Staining was performed in the dark for 20 min. LPS (2 μg mL⁻¹, Sigma) was used as positive control. Cells were processed by flow cytometry (FACS Canto II, BD Bioscience, CA, USA), and data were analyzed by FlowJo™ Software.

### Single-Cell Mass Cytometry Analysis (CyTOF)

Single-cell mass cytometry analysis was carried out using isolated PBMCs, obtained as previously reported. PBMCs were cultured in 6-well plates at a concentration of 4×10⁶ cells per well and treated with 50 μg/mL of the materials for 24 h at 37° C. Lipopolysaccharides (LPS) 0.5 μg/mL (Sigma-Aldrich, Missouri, USA), ethanol for cell biology (EtOH 70%) and untreated cells were used, respectively, as positive and negative controls.

Six hours before the end of the treatment, cells were incubated with Brefeldin A (Invitrogen, CA, USA) to a final concentration of 10 μg/mL. After the incubation time, cells were washed with a sterile solution of phosphate-buffered saline (PBS), EDTA 0.5 M and 5% of fetal calf serum (FCS). Cells were then combined using Cell-ID 20-Plex Pd Barcoding Kit (Fluidigm, CA, USA). The barcoded sample was stained with Cell-ID Cisplatin (Fluidigm, CA, USA) 1:1000, Maxpar Human Peripheral Blood Phenotyping and Human Intracellular Cytokine I Panel Kits (Fluidigm, CA, USA) following the manufacturer staining protocols.

In synthesis, to guarantee a uniform cell labelling with the palladium barcode, cells were fixed and permeabilized by means of 1× Fix I Buffer and 1× Barcode Perm Buffer. After the barcoding step, samples were pooled together and resuspended in Maxpar Cell Staining Buffer into a 5 mL polystyrene round-bottom tube.

The surface marker antibody cocktail (1:100 dilution for each antibody, final volume 800 μl) was added to the tube. The sample was mixed and incubated for 30 min at room temperature. After incubation, the sample was washed twice with Maxpar Cell Staining Buffer. Cells were then fixed by incubating the sample with 1 mL of 1.6% paraformaldehyde for 10 min. Subsequently, cells were washed twice with Maxpar Perm-S Buffer and centrifuged for 10 min at 1000×g. Cells were then resuspended in 400 μL of Maxpar Perm-S Buffer and incubated for other 30 min with cytoplasmic/secreted antibody cocktail (1:100 dilution for each antibody, final volume 800 μL). At the end of the incubation, cells were washed twice with Maxpar Cell Staining Buffer and stained overnight with Cell-ID Intercalator-Ir solution at the final concentration of 125 nM. Prior to data acquisition, the samples were washed twice with Maxpar Cell Staining Buffer, resuspended with 2 ml of Maxpar water and filtered using a 0.22 μm cell strainer cap to remove possible cell clusters or aggregates. Data were analyzed using mass cytometry platform CyTOF2(Fluidigm Corporation, CA, USA). Table 1 shows the antibody panel used for cell staining following the same procedure as above described.

### Gating Strategy and Data Analysis and Visualization

The CyTOF data analysis was carried out accordingly to the methods described by Orecchioni M et al. and Bendall et al. Briefly, normalized, background subtracted FCS files were uploaded into Cytobank for the analysis. The gating strategy excluded doublets, cell debris, and dead cells by means of Cell-ID Intercalator-Ir and LD. Specific PBMC subsets and subpopulations were assessed as reported in FIG. 10, in detail: T cells (CD45+ CD19− CD3+), T helper (CD45+ CD3+ CD4+), T cytotoxic (CD45+ CD3+ CD8+), T naive (CD45RA+ CD27+ CD38− HLADR−), T effector (CD45RA+ CD27− CD38− HLADR−), and activated (CD38+ HLADR+), B cells (CD45+ CD3− CD19+), B naive (HLADR+ CD27−), B memory (HLADR+ CD27+), plasma B (HLADR−CD38+), NK cells (CD45+ CD3− CD19− CD20− CD14− HLADR− CD38+ CD16+), Classical monocytes (CD45+ CD3− CD19− CD20− HLADR+ CD14+), Intermediate monocytes (CD45+ CD3− CD19− CD20− HLADR+ CD14dim CD16+) Non classical monocytes (CD45+ CD3− CD19− CD20− HLADR+ CD14− CD16+), and DC (CD45+ CD3− CD19− CD20− CD14− HLA− DR+ CD11c+ CD123−). The heat map visualization, realized with Cytobank, compared marker fluorescence of the treated populations with mean fluorescent intensity vs. the untreated control. tSNE tool was applied. tSNE, a cytometry analysis tool implemented in Cytobank, uses t-stochastic neighbor embedding (t-SNE) showing single cells in a two-or three-dimensional plot, according to their relationships. Nine cell surface markers were exploited in order to produce the tSNE map: CD3, CD4, CD8a, CD11c, CD14, CD16, CD19, CD20, CD123, and HLADR.

Cytokine data analysis was achieved using the tSNE tool. Plots showing the expression intensity of the analyzed cytokines (IFNγ, IL-2, IL4, IL-5, IL17a, IL17f, IL6, MIP1β, TNFα, Perforin, and GrB) and heat maps of mean marker expression ratio for all cytokines were realized.

### Cellular Detection of Material Uptake by IMC

Metal-labeled antibodies were provided by Fluidigm, from the standard CyTOF catalog (http://maxpar.fluidigm.com/product-catalog-metal.php). Metal-labeled antibody cocktails were prepared in 0.1% Tween-20, 1% BSA in PBS. All samples, were first blocked with 1% BSA and 0.2 mg mL⁻¹ mouse IgG Fc fragment (Thermo Scientific) in PBS for 30 min and then incubated with antibody cocktail for 1.5 h at RT, followed by washing with PBS and staining with DNA intercalator Ir-191/193 (Fluidigm) and CD45 (HI30)-89Y (Fluidigm) for 30 min. Slides were again washed with PBS and rinsed with ddH20 for 5 s and dried overnight at room temperature prior to IMC analysis.

ROIs of 500×500 μm undergo laser ablation aerosolizing a 1 μm² area/pulse (200 Hz), followed by ionization and quantification in the CyTOF Helios instrument. Ion mass data is collected for each pulse and processed to render images for each individual channel at 1 μm resolution, where the intensity of each pixel corresponds to the ion count value. Raw data were analyzed using Fluidigm MCD viewer program.

### Cellular Detection of Material Uptake by TEM

For Transmission Electron Microscopy (TEM) analysis, samples were fixed with 2.5% glutaraldehyde in 0.1M sodium cacodylate buffer pH 7.4 ON at 4° C. The samples were postfixed with 1% osmium tetroxide plus potassium ferrocyanide 1% in 0.1 M sodium cacodylate buffer for 1 h at 4° C. After three water washes, samples were dehydrated in a graded ethanol series and embedded in an epoxy resin (Sigma-Aldrich). Ultrathin sections (60-70 nm) were obtained with an Ultrotome V (LKB) ultramicrotome, counterstained with uranyl acetate and lead citrate and viewed with a Tecnai G² (FEI) transmission electron microscope operating at 100 kV. Images were captured with a Veleta (Olympus Soft Imaging System) digital camera.

### Luminex

To evaluate the impact of MXenes on cytokine release by PBMCs, cells were incubated for 24 h with 50 μg/mL of the materials. LPS, 2 μg/mL (Sigma) was used as positive control, while samples incubated with medium alone were used as negative controls. Supernatants were collected and analyzed by Luminex technology using Bio-Plex Pro Human Chemokine 40-plex Panel (Bio-Rad) to measure C-C Motif Chemokine Ligand (CCL) 21 (CCL21), chemokine (C-X-C motif) ligand (CXCL) 13 (CXCL13), CCL27, CXCL5, CCL11, CCL24, CCL26, C-X3-C Motif Chemokine Ligand 1 (CX3CL1), CXCL6, granulocyte macrophage-colony stimulating factor (GM-CSF), CXCL1, CXCL2, CCL1, interferon gamma (IFN-γ), interleukin (IL)-1β, IL-2, IL-4, IL-6, CXCL8, IL-10, IL-16, CXCL10, CXCL11, CCL2, CCL8, CCL7, CCL13, CCL22, macrophage migration inhibitory factor (MIF), CXCL9, CCL3, CCL15, CCL20, CCL19, CCL23, CXCL16, CXCL12, CCL17, CCL25 and tumor necrosis factor (TNF)-α. 5-parameter-Logistic regressions with a power low variance weighing were calculated for each cytokine standard with a recovery range of 70-130% using Bioplex Manager V6.2 (BioRad). Concentration falling within the recovery range, expressed in pg/ml were extrapolated from the median fluorescence intensity of each cytokine bead set. For analytes above or below the standard recovery ranges, upper and lower limits of quantification computed from the standard curves were substituted. Data were then Log2 transformed and compared across experiments by fitting a general ANOVA model with contrast between groups; p values were corrected using Benjamini and Hochberg false discovery rate, FDR; statistically significant p value cut-off was set at FDR p<0.05. Values out of range, “00R>” or “00R<”, were replaced, respectively, with the maximum or minimum value for the analyte across samples, indicated with (*), or, when not possible, with the upper (ULOQ) or lower limit of quantification (LLOQ) for that analyte, respectively.

### mRNAsequening

### RNA Extraction and QC

To evaluate the impact of MXenes on PBMCs, cells were incubated for 24 h with 50 μg/mL of the materials. Lipopolysaccharides (LPS 2 μg/mL, Sigma) and Concanavalin A (ConA, 10 μg/m, Sigma) were used as positive controls, while samples incubated with medium alone were used as negative controls. After treatment, the cell suspension was transferred from each well into RNase-free 1.5-mL tubes and cells were washed two times with 1 mL of PBS. Cells were then resuspended in 350 ul of RLT Buffer freshly additionated with 1% b-mercaptoethanol and stored at −80° C.

RNA was extracted using the RNAeasy Kit (Qiagen. The methodology has been followed detail in kit instruction. RNA was quantitated on a NanoDrop™ (ThermoFisher) and QCed using an Agilent 2100 Bioanalyzer (Agilent, Santa Clara, California, USA). All samples had a RIN>7.5.

### Normalization and Statistical Analysis

mRNA-sequencing was performed using QuantSeq 3′ mRNA-Seq Library Prep Kit FWD for Illumina (75 single-end) with a read depth of average 8.76 M, and average read alignment of 79.60%. Single samples were sequenced across four lanes, and the resulting FASTQ files were merged by sample. All FASTQ passed QC and were aligned to the reference genome GRChg38/hg19 using STAR 2.7.9a. BAM files were converted to a raw counts expression matrix using HTSeq-count. Then “betweenLaneNormalization” normalized data (using EDAseq omitting GC and transcript length correction (not applicable for 3′mRNA-seq)) was quantiled normalization and log2 transformed (total transcript mapped to genes=19,959 genes). All downstream analysis was performed using RStudio (Version 4.1., RStudio Inc.). Differential gene expression analysis was performed using Limma via Bioconductor package “limma v. 3.46.0” [PMID 25605792] with Benjamini-Hochberg (B-H) FDR, using different FDR p values cut-offs (e.g., 0.01, 0.05, and 0.1). In each comparison, genes with rows sum equal to zero were removed. Global transcriptional differences between samples were assessed by principal component analysis using the “prcomp” function. To illustrate the differentially expressed genes overlap between the conditions, R CRAN package “VennDiagram v. 1.6.20” was used. Differentially expressed genes were then plotted in a heatmap using Bioconductor package “ComplexHeatmap v. 2.6.2”. List of differentially expressed genes were uploaded to Ingenuity Pathway Analysis (IPA; Ingenuity Systems, Redwood City, CA) to detect modulated pathways (IPA canonical pathways).

### MXenes Quantification Determined by CyTOF Solution Mode

Dynamic range of ⁹³Nb, ⁹⁵Mo and ¹⁸¹Ta was determined by CyTOF solution mode. The dynamic range of MXenes was evaluated by mass cytometry CyTOF® Helios™ system at Fluidigm Canada, solution mode). Nb₄C₃—260 nm, Mo₂Ti₂C₃—240 nm, and Ta₄C₃—160 nm 50 μg/mL solutions were diluted 1:3 in order to create an ELISA curve (0.00, 0.0076, 0.023, 0.068, 0.2, 0.61, 1.85, 5.55, 16.6, and 50 μg/mL).

### Number of Nanomaterials Per Cell Calculation

The mean ⁹³Nb, ⁹⁵Mo and ¹⁸¹Ta ion intensity within a specific cell population measured by mass cytometry is addressed as the ‘mean dual counts’. This value is proportional to the number of ⁹³Nb, ⁹⁵Mo and ¹⁸¹Ta atoms per cell and it is the product of the integral over time of detector intensity multiplied by the dual count coefficient respectively of ⁹³Nb, ⁹⁵Mo and ¹⁸¹Ta.

The conversion of dual counts to the number of niobium, molybdenum and tantalum atoms per cell was determined using the calculations reported by Yang et al, Nat com, 2017 as follows:

Number of ⁹³Nb, ⁹⁵Mo and ¹⁸¹Ta atoms per cell=⁹³Nb, ⁹⁵Mo or ¹⁸¹Ta/¹⁹³Ir transmission factor

Since the transmission coefficient for ⁹³Nb, ⁹⁵Mo or ¹⁸¹Ta cannot be directly assessed in the cytometer, but can be measured for ¹⁹³Ir, characterized by a degree of ionization similar to the one of MXenes (Ir and MXenes have ionization energies of 8.9760 and 6.7589 (⁹³Nb), 7.0924 (⁹⁵Mo), 7.5496 (¹⁸¹Ta), eV, respectively). The ¹⁹³Ir transmission coefficient was calculated dividing the dual counts of ¹⁹³Ir detected for the instrument tuning solution by the number of ¹⁹³Ir atoms introduced in the 0.25 p.p.b. Ir tuning solution (Fluidigm CAT #201072) as reported below:

$$\begin{matrix}{}^{193} Ir transmission factor = \frac{{}^{193} Ir dual counts}{\begin{matrix}( Ir atoms introduced ) \times \\ ( {}^{193} Ir natural abundance )\end{matrix}} \\ = \frac{{}^{193} Ir dual counts}{{}^{193} Ir atoms introduced}\end{matrix} \qquad ( 2 )$$

Using the variables tuning solution Ir concentration (2.5×10⁻¹³ g μL⁻¹), flow rate (0.75 μL s⁻¹ for CyTOF2 or 0.5 μL s⁻¹ for Helios), the integration time (CyTOF2: 2.666 s; Helios: 4 s), the natural abundance of ¹⁹³Ir (0.627), Avogadro's number 6.02×10²³, and isotope mass (193 g mol⁻¹). Finally, the number of NPs per cell was calculated by the number of atoms per cell divided by the number of atoms per NP.

### Mice

Cd40^{G5/G5} and Cd40lg^{SrtA/Y}, CD4-Cre⁺, OT-II mice were housed in the SPF animal facility of the University of Padova, in accordance with institutional and ethical regulations. 5 to 12 weeks-old male and female mice were used in these experiments.

### Isolation of Splenic Dendritic Cells and Naïve CD4⁺ T Cells

To isolate dendritic cells, spleens were collected, incubated for 30 min at 37° C. in RPMI, 2% FBS, 20 mM HEPES, 400 U/ml type-IV collagenase (Sigma Aldrich) and disrupted to generate single-cell suspensions. Red-blood cells were lysed with ACK buffer (NH₄Cl 8.024 mg/l; KHCO₃ 1.001 mg/l; EDTA Na₂.2H₂O 3.722 mg/l), and the resulting cell suspensions were filtered through a 70 μm mesh into PBS supplemented with 0.5% BSA and 2 mM EDTA (PBE). DCs were obtained by magnetic cell separation (MACS) using anti-CD11c beads (Miltenyi Biotec), following the manufacturer's instructions.

To isolate naïve CD4⁺ T cells, spleens were harvested and single cell suspension were generated as described above. CD4⁺ T cells were then obtained by using the naïve CD4⁺ T cell isolation kit (Miltenyi Biotec) as per manufacturer's instructions.

### Labeling Immune Partnerships by SorTagging Intercellular Contacts (LIPSTIC)

Splenic DCs were isolated from Cd40^{G5/G5} mice as described above, seeded into round-bottom 96-well plates and treated with MXene cocktail (Nb₄C₃, Mo₂Ti₂C₃, and Ta₄C₃, 50 μg/mL each) for 24 h. After the incubation time, cells were washed two times to remove the nanomaterials dispersed in the media. Treated DCs were incubated with untreated naïve CD4⁺ T cells, freshly isolated from the spleens of Cd40lg^{SrtA/Y}, CD4-Cre⁺, OT-II mice (2×10⁵ total cells per well, 1:1 ratio). Medium was supplemented with 10 μM OTII peptide (OVA₃₂₉₋₃₃₇) and 10 μg/mL LPS, before being incubated for 24 h at 37° C. 30 minutes before the end of the incubation, the biotin-LPETG was added to each well at a final concentration of 10 μM in complete medium. At the end of the incubation, cells were washed three times with Maxpar PBS (Fluidigm) to remove excess biotin-LPETG substrate before CyTOF staining.

To allow dead cells discrimination, cells were resuspended in 500 ul Maxpar PBS containing 1 uM ¹⁹⁴Pt Cisplatin (Fluidigm), gently vortexed and incubated for 5 minutes at RT. Cells were then washed with Maxpar Cell Staining buffer and incubated with anti CD16/32 (BioXcell) for 10 minutes, at RT. After incubation, samples were stained for cell surface markers for 30 minutes at RT in Maxpar Cell Staining Buffer. The pool of antibodies used for the staining is reported in Table 2. Cells were then washed twice in Maxpar Cell Staining buffer, before being fixed by adding 16% paraformaldehyde to a final concentration of 1.6%, for 10 minutes at RT. DNA staining was performed by incubating the cells in Maxpar Fix and Perm buffer supplemented with 1:1000 ¹⁹¹Ir/¹⁹³Ir Cell-ID Intercalator (Fluidigm), for 18 h at 4° C. In the end, samples were acquired by using mass cytometry platform CyTOF2 (Fluidigm Corporation, CA, USA).

### In Vivo Biodistribution

For the in vivo biodistribution experiments 3 male C57BL/6J (cat. # 000664) mice per group were used. Mice were injected I.V. retro-orbitally with a 100 μL MXene cocktail (Nb₄C₃, Mo₂Ti₂C₃ and Ta₄C₃, 20 mg/g each in sterile PBS) or only sterile PBS. After 24 h mice were euthanized by CO₂ inhalation followed by blood withdrawal via cardiac puncture before further organ and tissue dissection. All experiments followed guidelines of the La Jolla Institute for Immunology (LJI) Animal Care and Use Committee. Approval for use of rodents was obtained from LJI according to criteria outlined in the Guide for the Care and Use of Laboratory Animals from the National Institutes of Health.

### Blood, Spleen, Lungs, and Livers where Harvested and Single Cell Suspension Isolated by Following Procedures

Blood was withdrawn via cardiac puncture and collected in EDTA-coated tubes (Sarstedt). Erythrocytes were lysed using 1× RBC lysis buffer (Biolegend) for 10 min at room temperature and the cell suspension was washed twice with PBS. Cells were kept in PBS with 2% FBS on until further staining and CyTOF analysis.

Spleens were homogenized through a 70 μm cell strainer (BD Biosciences), washed with 4° C. cold PBS and red blood cell lysis for 3 min at RT using 1× RBC lysis buffer (Biolegend). Splenocytes were washed with PBS and kept in PBS with 2% FCS, and kept on ice until further staining and CyTOF analysis.

Both lobes of a lung were rinsed with ice-cold PBS and transferred to a gentleMACS C tube (Miltenyi Biotec) and digested with 2 mg/mL collagenase D and 80 U/mL DNase I for 30 min at 37° C. on a gentleMACS Dissociator (Miltenyi Biotec). After digestion, lung cells were kept on ice in PBS with 2% FBS until further staining and CyTOF analysis.

The liver was dissected and homogenized through a 100 μm cell strainer (BD Biosciences). After washing in PBS, the liver cell pellet was resuspended in 10 mL of 37.5% percoll solution and centrifuged at 900×g for 25 min without acceleration and brake. The cell pellet was collected, washed with PBS, and kept on ice in PBS with 2% FBS until further staining and CyTOF analysis.

Table 1 shows the antibody panel used for cell staining following the same procedure as above described.

### MIBI-TOF

For the in vivo MIBI-TOF analysis 3 male C57BL/6J (cat. #000664) mice per group were used. Mice were injected I.V. retro-orbitally with a 100 μL MXene cocktail (Nb₄C₃, Mo₂Ti₂C₃, and Ta₄C₃, 20 mg/g each in sterile PBS) or only sterile PBS. After 24 h mice were euthanized by CO₂ inhalation followed by blood withdrawal via cardiac puncture before organ and tissue dissection. Spleen, Liver, Lungs and Kidneys were harvested and fixed for 24 h in a solution containing paraformaldehyde (PFA) 4%. After 24 h fixation, organs were washed and kept in 70% EtOH before paraffin embedding.

### Antibody Conjugation

A summary of antibodies, staining concentrations and conjugated metals can be found in 4. Metal conjugated primary antibodies were prepared as described previously,^{[45]} using antibody conjugation kits from Ionpath Inc.

### MIBI-TOF Staining

Staining was performed as previously described. Briefly, tissue sections (4 μm thick) were cut from FFPE tissue blocks and mounted on silanized-gold slides (Ionpath Inc.). Slide-tissue sections were baked at 70° C. for 20 min. Tissue sections were deparaffinized with 3 washes of fresh-xylene. Tissue sections were then rehydrated with successive washes of ethanol 100% (2×), 95% (2×), 80% (1×), 70% (1×), and distilled water. Washes were performed using a Leica ST4020 Linear Stainer (Leica Biosystems, Wetzlar, Germany) The sections were then immersed in epitope retrieval buffer (Antigen Retrieval Solution, Tris-EDTA, pH 9, abcam) and incubated at 97° C. for 40 min using Lab vision PT module (Thermofisher Scientific, Waltham, MA). Slides were washed with TBS with Tween 20 buffer (TBST, Ionpath Inc.). Sections were then blocked for 1 h with 3% (v/v) donkey serum (Sigma-Aldrich, St Louis, MO). Metal-conjugated antibody mix was prepared in 3% (v/v) donkey serum according to antibody concentrations in 4, and filtered using centrifugal filter, 0.1 μm PVDF membrane (Ultrafree-MC, Merck Millipore, Tullagreen Carrigtowhill, Ireland). Two panels of antibody mix were prepared: with the first, slides were incubated overnight at 4° C. in humid chamber; and with the second, slides were incubated the next morning for 1h at room temperature (according to incubation conditions for each antibody in 4). Slides were then washed twice 5 min in TBST wash buffer and fixed for 5 min in diluted glutaraldehyde solution 2% (Electron Microscopy Sciences, Hatfield, PA) in PBS-low barium. Tissue sections were then dehydrated with successive washes of Tris 0.1 M (pH 8.5), (3×), distilled water (2×), and ethanol 70% (1×), 80% (1×), 95% (2×), 100% (2×). Slides were immediately dried in a vacuum chamber for at least 1 h prior to imaging.

### Imaging and Image Processing

Imaging was performed using the MIBIscope system (Ionpath Inc.). MXene signal was detected for Niobium and Molybdenum at the ⁹³Nb and ⁹⁵Mo channels respectively. MXene signal at the Tantalum (¹⁸¹Ta) channel was also observed and matched the location of the signals in ⁹³Nb and ⁹⁵Mo. Notably, the slides used for MIBI-TOF are coated with Tantalum and as such any holes in the tissue, where bare slide is exposed, will give a high signal for Ta.^{[47]} This bare-slide signal may be difficult to decouple from the MXene signal if ¹⁸¹Ta will be used on its own. Additional work is needed to determine the best use of ¹⁸¹Ta as a reporter using the current methodology. Following image acquisition, output multi-dimensional TIFF images were processed for background subtraction, noise removal and aggregate removal using MAUI.

### Statistical Analysis

All values are expressed as mean±S.D. Comparison between groups was performed by one-way ANOVA, followed by a Tukey's post hoc multiple comparison where data was normally distributed. Data that did not follow the normal distribution were statistically analysed by Kruskall-Wallis ANOVA. Comparisons between two groups were performed using a two-tailed Student's t-test. A value of p<0.05 was considered significant.

### REFERENCES

[1] A. VahidMohammadi, J. Rosen, Y. Gogotsi, Science 2021, 372, eabf1581.
[2] Y. Gogotsi, B. Anasori, ACS Nano 2019, 13, 8491.
[3] M. Han, K. Maleski, C. E. Shuck, Y. Yang, J. T. Glazar, A. C. Foucher, K. Hantanasirisakul, A. Sarycheva, N. C. Frey, S. J. May, V. B. Shenoy, E. A. Stach, Y. Gogotsi, J Am Chem Soc 2020, 142, 19110.
[4] G. Deysher, C. E. Shuck, K. Hantanasirisakul, N. C. Frey, A. C. Foucher, K. Maleski, A. Sarycheva, V. B. Shenoy, E. A. Stach, B. Anasori, Y. Gogotsi, ACS Nano 2020, 14, 204.
[5] B. Anasori, M. R. Lukatskaya, Y. Gogotsi, Nat Rev Mater 2017, 2, 1.
[6] K. Li, M. Liang, H. Wang, X. Wang, Y. Huang, J. Coelho, S. Pinilla, Y. Zhang, F. Qi, V. Nicolosi, Y. Xu, Advanced Functional Materials 2020, 30, 2070306.
[7] Z. Zhang, S. Yang, P. Zhang, J. Zhang, G. Chen, X. Feng, Nat Commun 2019, 10, 2920.
[8] L. Fusco, A. Gazzi, G. Peng, Y. Shin, S. Vranic, D. Bedognetti, F. Vitale, A. Yilmazer, X. Feng, B. Fadeel, C. Casiraghi, L. G. Delogu, Theranostics 2020, 10, 5435.
[9] H. Lin, Y. Chen, J. Shi, Advanced Science 2018, 5, 1800518.
[10] N. Driscoll, B. Erickson, B. B. Murphy, A. G. Richardson, G. Robbins, N. V. Apollo, G. Mentzelopoulos, T. Mathis, K. Hantanasirisakul, P. Bagga, S. E. Gullbrand, M. Sergison, R. Reddy, J. A. Wolf, H. I. Chen, T. H. Lucas, T. R. Dillingham, K. A. Davis, Y. Gogotsi, J. D. Medaglia, F. Vitale, Sci Transl Med 2021, 13, eabf8629.
[11] A. Rafieerad, W. Yan, K. N. Alagarsamy, A. Srivastava, N. Sareen, R. C. Arora, S. Dhingra, Advanced Functional Materials 2021, 31, 2106786.
[12] A. Rafieerad, G. L. Sequiera, W. Yan, P. Kaur, A. Amiri, S. Dhingra, Journal of the Mechanical Behavior of Biomedical Materials 2020, 101, 103440.
[13] A. Gazzi, L. Fusco, A. Khan, D. Bedognetti, B. Zavan, F. Vitale, A. Yilmazer, L. G. Delogu, Front Bioeng Biotechnol 2019, 7, 295.
[14] M. A. Unal, F. Bayrakdar, L. Fusco, O. Besbinar, C. E. Shuck, S. Yalcin, M. T. Erken, A. Ozkul, C. Gurcan, O. Panatli, G. Y. Summak, C. Gokce, M. Orecchioni, A. Gazzi, F. Vitale, J. Somers, E. Demir, S. S. Yildiz, H. Nazir, J.-C. Grivel, D. Bedognetti, A. Crisanti, K. C. Akcali, Y. Gogotsi, L. G. Delogu, A. Yilmazer, Nano Today 2021, 38, 101136.
[15] X. Han, J. Huang, H. Lin, Z. Wang, P. Li, Y. Chen, Adv Healthc Mater 2018, 7, e1701394.
[16] C. Dai, Y. Chen, X. Jing, L. Xiang, D. Yang, H. Lin, Z. Liu, X. Han, R. Wu, ACS Nano 2017, 11, 12696.
[17] B. Lu, Z. Zhu, B. Ma, W. Wang, R. Zhu, J. Zhang, Small 2021, 17, 2100946.
[18] A. Rafieerad, W. Yan, G. L. Sequiera, N. Sareen, E. Abu-El-Rub, M. Moudgil, S. Dhingra, Advanced Healthcare Materials 2019, 8, 1900569.
[19] S. Iravani, R. S. Varma, ACS Biomater. Sci. Eng. 2021, 7, 1900.
[20] Y.-S. S. Yang, P. U. Atukorale, K. D. Moynihan, A. Bekdemir, K. Rakhra, L. Tang, F. Stellacci, D. J. Irvine, Nat Commun 2017, 8, 14069.
[21] B. Sahaf, A. Rahman, H. T. Maecker, S. C. Bendall, Methods Mol Biol 2020, 2055, 351.
[22] F. J. Hartmann, S. C. Bendall, Nat Rev Rheumatol 2020, 16, 87.
[23] M. H. Spitzer, G. P. Nolan, Cell 2016, 165, 780.
[24] L. Keren, M. Bosse, S. Thompson, T. Risom, K. Vijayaragavan, E. McCaffrey, D. Marquez, R. Angoshtari, N. F. Greenwald, H. Fienberg, J. Wang, N. Kambham, D. Kirkwood, G. Nolan, T. J. Montine, S. J. Galli, R. West, S. C. Bendall, M. Angelo, Science Advances 2019, 5, eaax5851.
[25] E. F. McCaffrey, M. Donato, L. Keren, Z. Chen, M. Fitzpatrick, V. Jojic, A. Delmastro, N. F. Greenwald, A. Baranski, W. Graf, M. Bosse, P. K. Ramdial, E. Forgo, D. V. Valen, M. van de Rijn, S. C. Bendall, N. Banaei, A. J. C. Steyn, P. Khatri, M. Angelo, 2020, 2020.06.08.140426.
[26] M. Angelo, S. C. Bendall, R. Finck, M. B. Hale, C. Hitzman, A. D. Borowsky, R. M. Levenson, J. B. Lowe, S. D. Liu, S. Zhao, Y. Natkunam, G. P. Nolan, Nat Med 2014, 20, 436.
[27] S. Chevrier, Y. Zurbuchen, C. Cervia, S. Adamo, M. E. Raeber, N. de Souza, S. Sivapatham, A. Jacobs, E. Bachli, A. Rudiger, M. Stüssi-Helbling, L. C. Huber, D. J. Schaer, J. Nilsson, O. Boyman, B. Bodenmiller, Cell Reports Medicine 2021, 2, 100166.
[28] M. Orecchioni, D. Bedognetti, L. Newman, C. Fuoco, F. Spada, W. Hendrickx, F. M. Marincola, F. Sgarrella, A. F. Rodrigues, C. Ménard-Moyon, G. Cesareni, K. Kostarelos, A. Bianco, L. G. Delogu, Nat Commun 2017, 8, 1109.
[29] M. Orecchioni, V. Bordoni, C. Fuoco, G. Reina, H. Lin, M. Zoccheddu, A. Yilmazer, B. Zavan, G. Cesareni, D. Bedognetti, A. Bianco, L. G. Delogu, Small 2020, 16, 2070117.
[30] L. Fusco, E. Avitabile, V. Armuzza, M. Orecchioni, A. Istif, D. Bedognetti, T. Da Ros, L. G. Delogu, Carbon 2020, 160, 390.
[31] M. Pescatori, D. Bedognetti, E. Venturelli, C. Ménard-Moyon, C. Bernardini, E. Muresu, A. Piana, G. Maida, R. Manetti, F. Sgarrella, A. Bianco, L. G. Delogu, Biomaterials 2013, 34, 4395.
[32] P. Foroozandeh, A. A. Aziz, Nanoscale Research Letters 2018, 13, 339.
[33] R. G. Mendes, A. Mandarino, B. Koch, A. K. Meyer, A. Bachmatiuk, C. Hirsch, T. Gemming, O. G. Schmidt, Z. Liu, M. H. Rümmeli, Nano Res. 2017, 10, 1980.
[34] A. Gupta, T. Sakthivel, S. Seal, Progress in Materials Science 2015, 73, 44.
[35] K. Huang, Z. Li, J. Lin, G. Han, P. Huang, Chem. Soc. Rev. 2018, 47, 5109.
[36] T. Porta Siegel, G. Hamm, J. Bunch, J. Cappell, J. S. Fletcher, K. Schwamborn, Mol Imaging Biol 2018, 20, 888.
[37] G. Pasqual, A. Chudnovskiy, J. M. J. Tas, M. Agudelo, L. D. Schweitzer, A. Cui, N. Hacohen, G. D. Victora, Nature 2018, 553, 496.
[38] E. Landhuis, Nature 2018, 557, 595.
[39] B. Fadeel, C. Bussy, S. Merino, E. Vázquez, E. Flahaut, F. Mouchet, L. Evariste, L. Gauthier, A. J. Koivisto, U. Vogel, C. Martin, L. G. Delogu, T. Buerki-Thurnherr, P. Wick, D. Beloin-Saint-Pierre, R. Hischier, M. Pelin, F. Candotto Carniel, M. Tretiach, F. Cesca, F. Benfenati, D. Scaini, L. Ballerini, K. Kostarelos, M. Prato, A. Bianco, ACS Nano 2018, 12, 10582.
[40] L. G. Delogu, S. M. Stanford, E. Santelli, A. Magrini, A. Bergamaschi, K. Motamedchaboki, N. Rosato, T. Mustelin, N. Bottini, M. Bottini, J Nanosci Nanotechnol 2010, 10, 5293.
[41] E. Avitabile, L. Fusco, S. Minardi, M. Orecchioni, B. Zavan, A. Yilmazer, M. Rauner, P. Pippia, E. Tasciotti, L. G. Delogu, Frontiers in Bioengineering and Biotechnology 2020, 8.
[42] V. Bordoni, G. Reina, M. Orecchioni, G. Furesi, S. Thiele, C. Gardin, B. Zavan, G. Cuniberti, A. Bianco, M. Rauner, L. G. Delogu, Nanoscale 2019, 11, 19408.
[43] J. Russier, V. León, M. Orecchioni, E. Hirata, P. Virdis, C. Fozza, F. Sgarrella, G. Cuniberti, M. Prato, E. Vázquez, A. Bianco, L. G. Delogu, Angewandte Chemie International Edition 2017, 56, 3014.
[44] S. C. Bendall, E. F. Simonds, P. Qiu, E. D. Amir, P. O. Krutzik, R. Finck, R. V. Bruggner, R. Melamed, A. Trejo, O. I. Ornatsky, R. S. Balderas, S. K. Plevritis, K. Sachs, D. Pe'er, S. D. Tanner, G. P. Nolan, Science 2011, 332, 687.
[45] S. C. Bendall, E. F. Simonds, P. Qiu, E. D. Amir, P. O. Krutzik, R. Finck, R. V Bruggner, R. Melamed, A. Trejo, O. I. Ornatsky, R. S. Balderas, S. K. Plevritis, K. Sachs, D. Pe'er, S. D. Tanner, G. P. Nolan, Science (New York, N.Y.) 2011, 332, 687.
[46] L. Keren, M. Bosse, D. Marquez, R. Angoshtari, S. Jain, S. Varma, S.-R. Yang, A. Kurian, D. Van Valen, R. West, S. C. Bendall, M. Angelo, Cell 2018, 174, 1373.
[47] A. Baranski, I. Milo, S. Greenbaum, J.-P. Oliveria, D. Mrdjen, M. Angelo, L. Keren, PLOS Computational Biology 2021, 17, e1008887.

TABLE 1
Antibody conjugation for CyTOF analysis. A summary of antibodies,
staining and conjugated metals used for CyTOF analysis.

| Target | Metal | Clone | Catalog # | Company |
|---|---|---|---|---|
| CD45 | 089Y | HI30 | 201325 | Fluidigm |
| CD196/CCR6 | 141Pr | G034E3 | 201325 | Fluidigm |
| CD235a/b | 141Pr | HIR2 | 201304 | Fluidigm |
| IL-4 | 142Nd | MP4-25D2 | 3142002B | Fluidigm |
| CD19 | 142Nd | HIB19 | 201304 | Fluidigm |
| CD123 (IL-3R) | 143Nd | 6H6 | 201325 | Fluidigm |
| IL-5 | 143Nd | TRFK5 | 201308 | Fluidigm |
| CD19 | 144Nd | HIB19 | 201325 | Fluidigm |
| IL-4 | 144Nd | MP4-25D2 | 201308 | Fluidigm |
| CD4 | 145Nd | RPA-T4 | 201325 | Fluidigm |
| CD8a | 146Nd | RPA-T8 | 201325 | Fluidigm |
| CD11c | 147Sm | Bu15 | 201325 | Fluidigm |
| CD20 | 147Sm | 2H7 | 201304 | Fluidigm |
| CD16 | 148Nd | 3G8 | 201325 | Fluidigm |
| CD45RO | 149Sm | UCHL1 | 201325 | Fluidigm |
| CD66 | 149Sm | CD66a-B1.1 | 201304 | Fluidigm |
| CD45RA | 150Nd | HI100 | 201325 | Fluidigm |
| MIP1β | 150Nd | D21-1351 | 201308 | Fluidigm |
| CD161 | 151Eu | HP-3G10 | 201325 | Fluidigm |
| CD123 | 151Eu | 6H6 | 201304 | Fluidigm |
| CD194/CCR4 | 152Sm | L291H4 | 201325 | Fluidigm |
| TNFα | 152Sm | Mab11 | 201308 | Fluidigm |
| CD25 | 153Eu | BC96 | 201325 | Fluidigm |
| CD27 | 154Sm | O323 | 201325 | Fluidigm |
| CD45 | 154Sm | HI30 | 201304 | Fluidigm |
| CD57 | 155Gd | HCD57 | 201325 | Fluidigm |
| CD183/CXCR3 | 156Gd | G025H7 | 201325 | Fluidigm |
| IL-6 | 156Gd | MQ2-13A5 | 201308 | Fluidigm |
| CD185/CXCR5 | 158Gd | J252D4 | 201325 | Fluidigm |
| IL-2 | 158Gd | MQ1-17H12 | 201308 | Fluidigm |
| GM-CSF | 159Tb | BVD2-21C11 | 3159008B | Fluidigm |
| CD11c | 159Tb | Bu15 | 201304 | Fluidigm |
| CD28 | 160Gd | CD28.2 | 201325 | Fluidigm |
| CD14 | 160Gd | M5E2 | 201304 | Fluidigm |
| CD38 | 161Dy | HB-7 | 201325 | Fluidigm |
| CD56 (NCAM) | 163Dy | NCAM16.2 | 201325 | Fluidigm |
| TCRgd | 164Dy | B1 | 201325 | Fluidigm |
| IL-17A | 164Dy | N49-653 | 201308 | Fluidigm |
| IFNg | 165Ho | B27 | 3165002B | Fluidigm |
| CD61 | 165Ho | VI-PL2 | 201304 | Fluidigm |
| IL-17F | 166Er | SHLR17 | 201308 | Fluidigm |
| CD27 | 167Er | O323 | 201304 | Fluidigm |
| CD294 (CRTH2) | 166Er | BM16 | 201325 | Fluidigm |
| CD197/CCR7 | 167Er | G043H7 | 201325 | Fluidigm |
| CD14 | 168Er | 63D3 | 201325 | Fluidigm |
| FNγ | 168Er | B27 | 201308 | Fluidigm |
| CD45RA | 169Tm | HI100 | 201304 | Fluidigm |
| CD3 | 170Er | UCHT1 | 201325 | Fluidigm |
| CD20 | 171Yb | 2H7 | 201325 | Fluidigm |
| Granzyme B | 171Yb | GB11 | 201308 | Fluidigm |
| CD66b | 172Yb | G10F5 | 201325 | Fluidigm |
| CD38 | 172Yb | HIT2 | 201304 | Fluidigm |
| HLA-DR | 173Yb | LN3 | 201325 | Fluidigm |
| IgD | 174Yb | IA6-2 | 201325 | Fluidigm |
| HLA-DR | 174Yb | L243 | 201304 | Fluidigm |
| TNFa | 175Lu | Mab11 | 3175023B | Fluidigm |
| Perforin B | 175Lu | D48 | 201308 | Fluidigm |
| CD127 (IL-Ra) | 176Yb | A019D5 | 201325 | Fluidigm |
| DNA | 191Ir | n/a | 201192B | Fluidigm |
| DNA | 193Ir | n/a | 201192B | Fluidigm |
| Cisplatin | 195Pt | n/a | 201064 | Fluidigm |
| Viability | | | | |

TABLE 2
Antibody conjugation for LIPSTIC analysis. A summary of antibodies,
staining and conjugated metals used for LIPSTIC analysis.

| Target | Metal | Clone | Catalog # | Company |
|---|---|---|---|---|
| I-A/I-E | 209Bi | M5/114.15.2 | 3209006B | Fluidigm |
| CD45R/B220 | 176Yb | RA36B2 | 3176002B | Fluidigm |
| CD11c | 162Dy | N418 | 3162017B | Fluidigm |
| CD69 | 143Nd | H1.2F3 | 3143004B | Fluidigm |
| CD4 | 172Yb | RM4-5 | 3172003B | Fluidigm |
| biotin | 150Nd | 1D4-C5 | 3150008B | Fluidigm |

TABLE 3
Antibody conjugation for in vivo biodistribution analysis.
A summary of antibodies, staining and conjugated metals
used for in vivo biodistribution analysis.

| Target | Metal | Clone | Catalog # | Company |
|---|---|---|---|---|
| CD45 | 89Y | 30-F11 | 3089005B | Fluidigm |
| Ly-6C | 150Nd | HK1.4 | 128002 | Biolegend |
| CD11c | 142Nd | N418 | 3142003B | Fluidigm |
| TCR-b | 143Nd | H57-597 | 3143010B | Fluidigm |
| CD11b | 148Nd | M1/70 | 101202 | Biolegend |
| CD19 | 145Nd | 6D5 | 115502 | Biolegend |
| CD8a | 146Nd | 53-6.7 | 3153012B | Fluidigm |
| NK1.1 | 165Ho | PK136 | 108702 | Biolegend |
| CD4 | 172Yb | RM4-5 | 3172003B | Fluidigm |
| CD117 | 173Yb | 2B8 | 3173004B | Fluidigm |
| Ly-6G | 141Pr | 1A8 | 127602 | Biolegend |
| DNA | 191Ir | n/a | 201192B | Fluidigm |
| DNA | 193Ir | n/a | 201192B | Fluidigm |
| Cisplatin | 195Pt | n/a | 201064 | Fluidigm |
| Viability | | | | |

TABLE 4
Antibody conjugation for MIBI-TOF analysis. A summary of antibodies, staining
concentrations and conjugated metals used for MIBI-TOF analysis.

| Antibody | | | | Conjugated | Concentration | Incubation |
|---|---|---|---|---|---|---|
| target | Clone | Source | Cat# | metal | (μg/mL) | conditions |
| CD3 | SP162 | abcam | ab245731 | 159Td | 1.67 | 4° C. overnight |
| CD31 | EPR17259 | abcam | ab225883 | 174yb | 2.5 | 4° C. overnight |
| CD74 | In1/CD74 | Biolegend | 151002 | 172Yb | 2.5 | 4° C. overnight |
| COL1A1 | E8F4L | CST | 72026 | 113In | 1.25 | 4° C. overnight |
| dsDNA | 3519 DNA | Ionpath | 708901 | 89Y | 0.375 | RT, 1 hour |
| F4/80 | D2S9R | CST | 70076 | 161Dy | 10 | 4° C. overnight |
| Na—K-ATPase | EP1845Y | Ionpath | 717603 | 176Yb | 1 | 4° C. overnight |
| SMA | SP171 | abcam | ab242395 | 115Ln | 0.5 | RT, 1 hour |
| Vimentin | D21H3 | CST | 5741 | 168Er | 1.25 | 4° C. overnight |

### ASPECTS

The following Aspects are illustrative only and do not limit the scope of the present disclosure or the appended claims. Any part or parts of any one or more Aspects can be combined with any part or parts of any one or more other Aspects.

Aspect 1. A method, comprising: tagging at least one cell with a MXene, the cell optionally being an immune cell; and detecting at least one component of the MXene using one or more of single-cell mass cytometry by time-of-flight (CyTOF), imaging mass cytometry (IMC), and ion beam imaging (MIBI-TOF). Tagging can comprise, for example, placing the MXene within the cell, associating the MXene with the exterior of the cell, or both. The detected component of the MXene can be, for example, an atom or atoms of the MXene. Such an atom can be an isotope.

MXenes are derived from the selective etching of their corresponding Mₙ₊₁AXₙ (MAX) phases. They are represented by the formula, Mₙ₊₁XₙTₓ, where M denotes an early transition metal (Ti, Nb, V, Hf, Ta, etc.), X is carbon and/or nitrogen, Tx indicates the surface terminations (O, OH, F, etc.), and n=1, 2, 3, or 4. One can modulate MXenes by a multi-modular chemistry design based on a biological selection of the elements and the appropriate ratios of M or X elements. One can select elements for the materials according to the mass cytometry detection range, which is between 75 and 209 Da in some embodiments, by avoiding toxic and radioactive elements. One can also provide MXene tags that have different ratios of detectable elements. One can also provide MXene tags derived from MAX phases constituted by pure isotopes of detectable elements.

Aspect 2. The method of Aspect 1, wherein (1) the MXene exhibits a MXene detection range detectable by the one or more of single-cell mass cytometry by time-of-flight (CyTOF), imaging mass cytometry (IMC), and ion beam imaging by time-of-flight (MIBI-TOF), wherein (2) the cell comprises a tag that is exhibits a tag detection range detectable by the one or more of single-cell mass cytometry by time-of-flight (CyTOF), imaging mass cytometry (IMC), and ion beam imaging by time-of-flight (MIBI-TOF), and (3) wherein the MXene detection range is free of overlap with the tag detection range, the tag optionally comprising a metal-tagged antibody.

Aspect 3. The method of any one of Aspects 1 or 2, further comprising applying a classification to the least one cell according to a degree of detection of least one component of the MXene using any one or more of single-cell mass cytometry by time-of-flight (CyTOF), imaging mass cytometry (IMC), and ion beam imaging by time-of-flight (MIBI-TOF).

Aspect 4. The method of Aspect 3, wherein the classification relates to a location of the cell, a physiological characteristic of the cell, or both. One can also utilize the disclosed technology to determine a distribution of MXene within a cell. The classification can be based on a MXene-based barcode, for example, a MXene that encodes information regarding one, two, or more characteristics of the cell.

Aspect 5. The method of any one of Aspects 1 to 4, wherein the detecting is by mass cytometry by time of flight (CyTOF).

Aspect 6. The method of any one of Aspects 1 to 4, wherein the detecting is by mass cytometry by imaging mass cytometry (IMC).

Aspect 7. The method of any one of Aspects 1 to 4, wherein the detecting is by mass cytometry by ion beam imaging by time-of-flight (MIBI-TOF).

Aspect 8. The method of any one of Aspects 1 to 7, wherein the cell is comprised in a tissue. Such tissue can be, for example, muscular tissue, bone, neural tissue, and the like.

Aspect 9. The method of Aspect 8, further comprising applying a classification to the tissue according to a degree of detection of least one component of the MXene using any one or more of single-cell mass cytometry by time-of-flight (CyTOF), imaging mass cytometry (IMC), and ion beam imaging by time-of-flight (MIBI-TOF).

Aspect 10. The method of Aspect 9, wherein the classification relates to a location of the tissue, a physiological characteristic of the tissue, or both.

Aspect 11. The method of any one of Aspects 1 to 10, wherein the MXene has an atomic mass of from 75 to 209.

Aspect 12. The method of any one of Aspects 1 to 10, wherein the MXene comprises any one or more of Nb₄C₃, Mo₂Ti₂C₃ and Ta₄C₃.

Aspect 13. The method of any one of Aspects 1 to 12, wherein the detection is in at least one of the niobium (⁹³Nb), molybdenum (e.g., ^{92, 94, 95, 96 97, 98, 100}Mo) and tantalum (¹⁸⁰⁻¹⁸¹Ta) channels.

Aspect 14. A system, the system configured to perform the method of any one of Aspects 1 to 13.

Aspect 15. A system, the system comprising: a cell tagged with an amount of a MXene; and a detection train configured to perform on the cell at least one of time of flight (CyTOF), imaging mass cytometry (IMC), and ion beam imaging by time-of-flight (MIBI-TOF) that detects the MXene.

Aspect 16. A method, the method comprising: tagging a population of cells with at least one MXene; and processing the population of cells with at least one of time-of-flight (CyTOF), imaging mass cytometry (IMC), and ion beam imaging by time-of-flight (MIBI-TOF) that detects the at least MXene; and relating the detection of the at least one MXene to a characteristic of the population of cells.

The disclosed methods can also include tagging the population of cells with a tag, the tag and the MXene being detectable in non-overlapping ranges by the at least one of time of flight (CyTOF), imaging mass cytometry (IMC), and ion beam imaging by time-of-flight (MIBI-TOF). In this way, one can use a MXene and a separate tag to obtain information regarding a cell or even a population of cells.

As described elsewhere herein, the disclosed technology can be used in a range of applications, including labeling, detection, identification, and tracking of cells, in addition to other applications such as cell barcoding systems, spatially resolved biological targets in tissues, and detection of biological markers with single-cell resolution. In particular, the disclosed technology can be used in, e.g., immunology and regenerative medicine for (i) multi-imaging agents for cell-tracking and (ii) detection of weakly expressed antigens or rare cell populations.

The disclosed technology can be used to determine whether a certain cell process is occurring (or not occurring), the distribution of cells in a tissue, to determine uptake of the MXene tags, and the like. One can evaluate docking of MXene tags experimentally and/or via simulation. As but one example, the disclosed technology is compatible with and can be used with commercial panels of antibodies (for example, metal-tagged antibodies for CyTOF), which provides an efficient way of tracking the materials together with several biological information; the method can also be used to conjugate drugs. The disclosed technology also allows material tracking on a high number of cell types at the same time, within tissues and at single-cell level. The disclosed technology can also be adapted for other 2D materials useful for biomedical applications based on their masses.

1. A method, comprising:
tagging at least one cell with a MXene, the cell optionally being an immune cell; and
detecting at least one component of the MXene using one or more of single-cell mass cytometry by time-of-flight (CyTOF), imaging mass cytometry (IMC), and ion beam imaging by time-of-flight (MIBI-TOF).

2. The method of claim 1, wherein (1) the MXene exhibits a MXene detection range detectable by the one or more of single-cell mass cytometry by time-of-flight (CyTOF), imaging mass cytometry (IMC), and ion beam imaging by time-of-flight (MIBI-TOF), wherein (2) the cell comprises a tag that is exhibits a tag detection range detectable by the one or more of single-cell mass cytometry by time-of-flight (CyTOF), imaging mass cytometry (IMC), and ion beam imaging by time-of-flight (MIBI-TOF), and (3) wherein the MXene detection range is free of overlap with the tag detection range, the tag optionally comprising a metal-tagged antibody.

3. The method of claim 1, further comprising applying a classification to the least one cell according to a degree of detection of least one component of the MXene using any one or more of single-cell mass cytometry by time-of-flight (CyTOF), imaging mass cytometry (IMC), and ion beam imaging by time-of-flight (MIBI-TOF).

4. The method of claim 3, wherein the classification relates to a location of the cell, a physiological characteristic of the cell, or both.

5. The method of claim 1, wherein the detecting is by mass cytometry by time-of-flight (CyTOF).

6. The method of claim 1, wherein the detecting is by mass cytometry by imaging mass cytometry (IMC).

7. The method of claim 1, wherein the detecting is by mass spectrometry by ion beam imaging by time-of-flight (MIBI-TOF).

8. The method of claim 1, wherein the cell is comprised in a tissue.

9. The method of claim 8, further comprising applying a classification to the tissue according to a degree of detection of least one component of the MXene using any one or more of single-cell mass cytometry by time-of-flight (CyTOF), imaging mass cytometry (IMC), and ion beam imaging by time-of-flight (MIBI-TOF).

10. The method of claim 9, wherein the classification relates to a location of the tissue, a physiological characteristic of the tissue, or both.

11. The method of claim 1, wherein the MXene has an atomic mass of from 75 to 209.

12. The method of claim 1, wherein the MXene comprises one or more of Nb₄C₃, Mo₂Ti₂C₃ and Ta₄C₃.

13. The method of claim 1, wherein the detection is in at least one of the niobium (⁹³Nb), molybdenum (^{92, 94, 95, 96 97, 98, 100}Mo) and tantalum (¹⁸⁰⁻¹⁸¹Ta) channels.

14. A system, the system configured to perform the method of claim 1.

15. A system, the system comprising:
a cell tagged with an amount of a MXene; and
a detection train configured to perform on the cell at least one of time-of-flight (CyTOF), imaging mass cytometry (IMC), and ion beam imaging by time-of-flight (MIBI-TOF) that detects the MXene.

16. A method, the method comprising:
tagging a population of cells with at least one MXene; and
processing the population of cells with at least one of time-of-flight (CyTOF), imaging mass cytometry (IMC), and ion beam imaging by time-of-flight (MIBI-TOF) that detects the at least MXene; and
relating the detection of the at least one MXene to a characteristic of the population of cells.
